SEQUENCE LISTING (42 sequences)

SEQ ID NO: 1 - DNA, 3700 bp, Homo sapiens
  CDS (236)..(3178)
gaattccggagttgctgccgctgccttcagcaagacgctgctctgaggcggggagggcgccgcgtcctgagcgcgcggcccagcgtcacggcggcggcggcggcggctcctccttggacccccggagctccccgcgccgcgagcagctggccccaggcccctagagccccgagagctccgagagctccgctcggcgtcccgcgcgcctccctgccgctcccgccccgggctggcgatgctgcgccgccccgctcccgagctggccccggccgcccggctgctgctggccgggctgctgtgcggcggcggggtctgggccgcgcgagttaacaagcacaagccctggctggagcccacctaccacggcatagtcacagagaacgacaacaccgtgctcctcgaccccccactgatcgcgctggataaagatgcgcctctgcgatttgcagagagttttgaggtgacagtcaccaaagaaggtgagatttgtggatttaaaattcacgggcagaatgtcccctttgatgcagtggtagtggataaatccactggtgagggagtcattcgctccaaagagaaactggactgtgagctgcagaaagactattcattcaccatccaggcctatgattgtgggaagggacctgatggcaccaacgtgaaaaagtctcataaagcaactgttcatattcaggtgaacgacgtgaatgagtacgcgcccgtgttcaaggagaagtcctacaaagccacggtcatcgaggggaagcagtacgacagcattttgagggtggaggccgtggatgccgactgctcccctcagttcagccagatttgcagctacgaaatcatcactccagacgtgccctttactgttgacaaagatggttatataaaaaacacagagaaattaaactacgggaaagaacatcaatataagctgaccgtcactgcctatgactgtgggaagaaaagagccacagaagatgttttggtgaagatcagcattaagcccacctgcacccctgggtggcaaggatggaacaacaggattgagtatgagccgggcaccggcgcgttggccgtctttccaaatatccacctggagacatgtgacgagccagtcgcctcagtacaggccacagtggagctagaaaccagccacatagggaaaggctgcgaccgagacacctactcagagaagtccctccaccggctctgtggtgcggccgcgggcactgccgagctgctgccatccccgagtggatccctcaactggaccatgggcctgcccaccgacaatggccacgacagcgaccaggtgtttgagttcaacggcacccaggcagtgaggatcccggatggcgtcgtgtcggtcagccccaaagagccgttcaccatctcggtgtggatgagacatgggccattcggcaggaagaaggagacaattctttgcagttctgataaaacagatatgaatcggcaccactactccctctatgtccacgggtgccggctgatcttcctcttccgtcaggatccttctgaggagaagaaatacagacctgcagagttccactggaagttgaatcaggtctgtgatgaggaatggcaccactacgtcctcaatgtagaattcccgagtgtgactctctatgtggatggcacgtcccacgagcccttctctgtgactgaggattacccgctccatccatccaagatagaaactcagctcgtggtgggggcttgctggcaagagttttcaggagttgaaaatgacaatgaaactgagcctgtgactgtggcctctgcaggtggcgacctgcacatgacccagtttttccgaggcaatctggctggcttaactctccgttccgggaaactcgcggataagaaggtgatcgactgtctgtatacctgcaaggaggggctggacctgcaggtcctcgaagacagtggcagaggcgtgcagatccaagcacaccccagccagttggtattgaccttggagggagaagacctcggggaattggataaggccatgcagcacatctcgtacctgaactcccggcagttccccacgcccggaattcgcagactcaaaatcaccagcacaatcaagtgttttaacgaggccacctgcatttcggtccccccggtagatggctacgtgatggttttacagcccgaggagcccaagatcagcctgagtggcgtccaccattttgcccgagcagcttctgaatttgaaagctcagaaggggtgttccttttccctgagcttcgcatcatcagcaccatcacgagagaagtggagcctgaaggggacggggctgaggaccccacagttcaagaatcactggtgtccgaggagatcgtgcacgacctggatacctgtgaggtcacggtggagggagaggagctgaaccacgagcaggagagcctggaggtggacatggcccgcctgcagcagaagggcattgaagtgagcagctctgaactgggcatgaccttcacaggcgtggacaccatggccagctacgaggaggttttgcacctgctgcgctatcggaactggcatgccaggtccttgcttgaccggaagtttaagctcatctgctcagagctgaatggccgctacatcagcaacgaatttaaggtggaggtgaatgtaatccacacggccaaccccatggaacacgccaaccacatggctgcccagccacagttcgtgcacccggaacaccgctcctttgttgacctgtcaggccacaacctggccaacccccacccgttcgcagtcgtccccagcactgcgacagttgtgatcgtggtgtgcgtcagcttcctggtgttcatgattatcctgggggtatttcggatccgggccgcacatcggcggaccatgcgggatcaggacaccgggaaggagaacgagatggactgggacgactctgccctgaccatcaccgtcaaccccatggagacctatgaggaccagcacagcagtgaggaggaggaggaagaggaagaggaagaggaaagcgaggacggcgaagaagaggatgacatcaccagcgccgagtcggagagcagcgaggaggaggagggggagcagggcgacccccagaacgcaacccggcagcagcagctggagtgggatgactccaccctcagctactgacccgtgcccccggccacctcggtttctgctttcgaagactctgctgccatccgttctcccagtcccaagggtccacgatgtacaaagtcatttcggccagtaggtgtgcagacccctccccgccacgatcgtcgctgtgcttggtgtgtaggaccctaggctccccgcccaccctctgcctggtcgcgctcttcagtcccacgaggagctgacacgtcctctctggccgccatccggctcgcacaggggcctcccagcgcctcaggccccgcgtttgtgtctggagtctccccccggggagaggacactggcccctcgcactccagaaaagccatgccagctgggctcgttgacaaagggtaaaacatgctcatcccacccggtaattcatttttttcttttttttaaaaaaagtttttatttttttccaaactagtgcatgtattaaataatgggcaggggggggggtatgttttaggatgattaactggactttttaatattttttgtaaaataaaa

SEQ ID NO: 2 - protein, 981 aa, Homo sapiens
MLRRPAPELAPAARLLLAGLLCGGGVWAARVNKHKPWLEPTYHGIVTENDNTVLLDPPLIALDKDAPLRFAESFEVTVTKEGEICGFKIHGQNVPFDAVVVDKSTGEGVIRSKEKLDCELQKDYSFTIQAYDCGKGPDGTNVKKSHKATVHIQVNDVNEYAPVFKEKSYKATVIEGKQYDSILRVEAVDADCSPQFSQICSYEIITPDVPFTVDKDGYIKNTEKLNYGKEHQYKLTVTAYDCGKKRATEDVLVKISIKPTCTPGWQGWNNRIEYEPGTGALAVFPNIHLETCDEPVASVQATVELETSHIGKGCDRDTYSEKSLHRLCGAAAGTAELLPSPSGSLNWTMGLPTDNGHDSDQVFEFNGTQAVRIPDGVVSVSPKEPFTISVWMRHGPFGRKKETILCSSDKTDMNRHHYSLYVHGCRLIFLFRQDPSEEKKYRPAEFHWKLNQVCDEEWHHYVLNVEFPSVTLYVDGTSHEPFSVTEDYPLHPSKIETQLVVGACWQEFSGVENDNETEPVTVASAGGDLHMTQFFRGNLAGLTLRSGKLADKKVIDCLYTCKEGLDLQVLEDSGRGVQIQAHPSQLVLTLEGEDLGELDKAMQHISYLNSRQFPTPGIRRLKITSTIKCFNEATCISVPPVDGYVMVLQPEEPKISLSGVHHFARAASEFESSEGVFLFPELRIISTITREVEPEGDGAEDPTVQESLVSEEIVHDLDTCEVTVEGEELNHEQESLEVDMARLQQKGIEVSSSELGMTFTGVDTMASYEEVLHLLRYRNWHARSLLDRKFKLICSELNGRYISNEFKVEVNVIHTANPMEHANHMAAQPQFVHPEHRSFVDLSGHNLANPHPFAVVPSTATVVIVVCVSFLVFMIILGVFRIRAAHRRTMRDQDTGKENEMDWDDSALTITVNPMETYEDQHSSEEEEEEEEEEESEDGEEEDDITSAESESSEEEEGEQGDPQNATRQQQLEWDDSTLSY

SEQ ID NO: 3 - DNA, 4421 bp, Homo sapiens
  5′UTR (1)..(10)
  CDS (11)..(2875)
  3′UTR (2876)..(4421)
tgctgcgaggatgctgcctgggcggctgtgctgggtgccgctcctgctggcgctgggcgtggggagcggcagcggcggtggcggggacagccggcagcgccgcctcctcgcggctaaagtcaataagcacaagccatggatcgagacttcatatcatggagtcataactgagaacaatgacacagtcattttggacccaccactggtagccctggataaagatgcaccggttccttttgcaggggaaatctgtgcgttcaagatccatggccaggagctgccctttgaggctgtggtgctcaacaagacatcaggagagggccggctccgtgccaagagccccattgactgtgagttgcagaaggagtacacattcatcatccaggcctatgactgtggtgctgggccccacgagacagcctggaaaaagtcacacaaggccgtggtccatatacaggtgaaggatgtcaacgagtttgctcccaccttcaaagagccagcctacaaggctgttgtgacggagggcaagatctatgacagcattctgcaggtggaggccattgacgaggactgctccccacagtacagccagatctgcaactatgaaatcgtcaccacagatgtgccttttgccatcgacagaaatggcaacatcaggaacactgagaagctgagctatgacaaacaacaccagtatgagatcctggtgaccgcctacgactgtggacagaagcccgctgctcaggacaccctggtgcaggtggatgtgaagccagtttgcaagcctggctggcaagactggaccaagaggattgagtaccagcctggctccgggagcatgcccctgttccccagcatccacctggagacgtgcgatggagccgtgtcttccctccagatcgtcacagagctgcagactaattacattgggaagggttgtgaccgggagacctactctgagaaatcccttcagaagttatgtggagcctcctctggcatcattgacctcttgccatcccctagcgctgccaccaactggactgcaggactgctggtggacagcagtgagatgatcttcaagtttgacggcaggcagggtgccaaaatccccgatgggattgtgcccaagaacctgaccgatcagttcaccatcaccatgtggatgaaacacggccccagccctggtgtgagagccgagaaggaaaccatcctctgcaactcagacaaaaccgaaatgaaccggcatcactatgccctgtatgtgcacaactgccgcctcgtctttctcttgcggaaggacttcgaccaggctgacacctttcgccccgcggagttccactggaagctggatcagatttgtgacaaagagtggcactactatgtcatcaatgtggagtttcctgtggtaaccttatacatggatggagcaacatatgaaccatacctggtgaccaacgactggcccattcatccatctcacatagccatgcaactcacagtcggcgcttgttggcaaggaggagaagtcaccaaaccacagtttgctcagttctttcatggaagcctggccagtctcaccatccgccctggcaaaatggaaagccagaaggtgatctcctgcctgcaggcctgcaaggaagggctggacattaattccttggaaagccttggccaaggaataaagtatcacttcaacccctcgcagtccatcctggtgatggaaggtgacgacattgggaacattaaccgtgctctccagaaagtctcctacatcaactccaggcagttcccaacggcgggtgtgcggcgcctcaaagtatcctccaaagtccagtgctttggggaagacgtatgcatcagtatccctgaggtagatgcctatgtgatggtcctccaggccatcgagccccggatcaccctccggggcacagaccacttctggagacctgctgcccagtttgaaagtgccaggggagtgaccctcttccctgatatcaagattgtgagcaccttcgccaaaaccgaagcccccggggacgtgaaaaccacagaccccaaatcagaagtcttagaggaaatgcttcataacttagatttctgtgacattttggtgatcggaggggacttggacccaaggcaggagtgcttggagctcaaccacagtgagctccaccaacgacacctggatgccactaattctactgcaggctactccatctacggtgtgggctccatgagccgctatgagcaggtgctacatcacatccgctaccgcaactggcgtccggcttcccttgaggcccggcgtttccggattaagtgctcagaactcaatgggcgctacactagcaatgagttcaacttggaggtcagcatccttcatgaagaccaagtctcagataaggagcatgtcaatcatctgattgtgcagcctcccttcctccagtctgtccatcatcctgagtcccggagtagcatccagcacagttcagtggtcccaagcattgccacagtggtcatcatcatctccgtgtgcatgcttgtgtttgtcgtggccatgggtgtgtaccgggtccggatcgcccaccagcacttcatccaggagactgaggctgccaaggaatctgagatggactgggacgattctgcgctgactatcacagtcaaccccatggagaaacatgaaggaccagggcatggggaagatgagactgagggagaagaggaggaagaagccgaggaagaaatgagctccagcagtggctctgacgacagcgaagaggaggaggaggaggaagggatgggcagaggcagacatgggcagaatggagccaggcaagcccagctggagtgggatgactccaccctcccctactagtgcccaggggtctgctgcctggcccacatgtcccttttgtaaaccctgacccagtgtatgcccatgtctatcatacctcacctctgatgtctgtgacatgtctgggaaggccttctccagcttcctggagcccaccctttaagccttgggcactccctgtgtttcatccatggggaagttccaagaagcccagcatggccatcagtgaggacttcagggtagactttgtcctgtagcctccacttctgccctaagttccccagcatcctgactacctgtctgcagagtttgcctttgttttttcctgcagggaagaaggcccacctttgtgtcactcacctccccaggctcagagtccccaaggccctggggttccaactcactgtgcgtctcctccacacagaccagtaggttctcctatgctgactccaggttgcttcatacaaggagggtggttgaacttcacacacgtaaggtcttagtgcttaacagtttaaaggaaagtccttgttgaggcagaactaagtttacagggaaaggtacacacattctctctctctctctctctctgtctatctagttccccagcttggagagcctttccccttgcttctttctgaggccatataagcttataagaaaagtcccaaaccaagaataggtccttggccacaagcagggtctgatcccccatcagagctatctgagcctgcctgtctgggcacctgctgcaaccatgcagctaccctgccaggggcactcagcaaacagaaccacagggcccaggaggcattccacacaggcactgccccaggacaacacaacaaggacagtcacaacaaggacaacaaggacacaacacaacacacaacaaggacagtcacaacaagcctagagccagaaagcagatggaaatgctaatgaggtcaaacgtaggcttcatggtgggtggagtgggggtggctgggctcccccaggacagaggggaccctgaggttggcaaggctctcaccactcagccttatggtcccttatctcctatcttccctcttgagaaaatacacgctttctgcatgtattagaaacgcacgagctccaccaagtctacaatgaaagtttgaaatttaactgcaaggaattagaagcatatttgcaatcattgcagcttcttctttcttctgctcataaaaggaggaacactttagatagagggcaaatatatctgaaaacctaatttctttctttttttgataaggaaatcttttccatctccatcctaacatgcacaacctgtgaagagaattgtttctatagtaactggtctgtgatcttttgtggccaagagaatagcaggcaagaattagggccttgacagaatttccacgaagctctgagaacatgtttgtttcgaatgtctgattcctctttgtcatcaatgtgtatgctctgtccccatccttcactcctcctcaagctcacaccaattggtttggcacaggcacagagctggtccctagttaagtggcatttatgttaaaaaaaa

SEQ ID NO: 4 - protein, 955 aa, Homo sapiens
MLPGRLCWVPLLLALGVGSGSGGGGDSRQRRLLAAKVNKHKPWIETSYHGVITENNDTVILDPPLVALDKDAPVPFAGEICAFKIHGQELPFEAVVLNKTSGEGRLRAKSPIDCELQKEYTFIIQAYDCGAGPHETAWKKSHKAVVHIQVKDVNEFAPTFKEPAYKAVVTEGKIYDSILQVEAIDEDCSPQYSQICNYEIVTTDVPFAIDRNGNIRNTEKLSYDKQHQYEILVTAYDCGQKPAAQDTLVQVDVKPVCKPGWQDWTKRIEYQPGSGSMPLFPSIHLETCDGAVSSLQIVTELQTNYIGKGCDRETYSEKSLQKLCGASSGIIDLLPSPSAATNWTAGLLVDSSEMIFKFDGRQGAKIPDGIVPKNLTDQFTITMWMKHGPSPGVRAEKETILCNSDKTEMNRHHYALYVHNCRLVFLLRKDFDQADTFRPAEFHWKLDQICDKEWHYYVINVEFPVVTLYMDGATYEPYLVTNDWPIHPSHIAMQLTVGACWQGGEVTKPQFAQFFHGSLASLTIRPGKMESQKVISCLQACKEGLDINSLESLGQGIKYHFNPSQSILVMEGDDIGNINRALQKVSYINSRQFPTAGVRRLKVSSKVQCFGEDVCISIPEVDAYVMVLQAIEPRITLRGTDHFWRPAAQFESARGVTLFPDIKIVSTFAKTEAPGDVKTTDPKSEVLEEMLHNLDFCDILVIGGDLDPRQECLELNHSELHQRHLDATNSTAGYSIYGVGSMSRYEQVLHHIRYRNWRPASLEARRFRIKCSELNGRYTSNEFNLEVSILHEDQVSDKEHVNHLIVQPPFLQSVHHPESRSSIQHSSVVPSIATVVIIISVCMLVFVVAMGVYRVRIAHQHFIQETEAAKESEMDWDDSALTITVNPMEKHEGPGHGEDETEGEEEEEAEEEMSSSSGSDDSEEEEEEEGMGRGRHGQNGARQAQLEWDDSTLPY

SEQ ID NO: 5 - DNA, 3187 bp, Homo sapiens
  5′UTR (1)..(275)
  CDS (276)..(3143)
ctgcagtagcggggttggggtgggagtgagagagtgaggacgctgggctgggggaaacgggaagccgctgcaagtccaccgcctcagctacccagattgggatctgcccaggcccgctttatggactagtgtgggcggcaggctcctttccgtccctgccctgctgtaccccgctccttggagaccccctgtatccctcccgcaaggtggaatccgcaggctggaggctcccaggggaggcaaacgcctggccctgccctgccccacgccgcaccatgaccctcctgctgctgccccttctgctggcctctctgctcgcgtcctgctcctgtaacaaagccaacaagcacaagccatggattgaggcagagtaccagggcatcgtcatggagaatgacaacacggtcctactgaatccaccactctttgccttggacaaggatgccccgctgcgctatgcaggtgagatctgcggcttccggctccatgggtctggggtgccctttgaggctgtgatccttgacaaggcgacaggagaggggctgatccgggccaaggagcctgtggactgcgaggcccagaaggaacacaccttcaccatccaggcctatgactgtggcgagggccccgacggggccaacaccaagaagtcccacaaggccactgtgcatgtgcgggtcaacgatgtgaacgagtttgccccagtgtttgtggaacggctgtatcgtgcggctgtgacagaggggaagctgtacgatcgcatcctgcgggtggaagccattgacggtgactgctccccccagtacagccagatctgctactatgagattctcacacccaacacccctttcctcattgacaatgacgggaacattgagaacacagagaagctgcagtacagtggtgagaggctctataagtttacagtgacagcttatgactgtgggaagaagcgggcagcagatgatgctgaggtggagattcaggtgaagcccacctgtaaacccagctggcaaggctggaacaaaaggatcgaatatgcaccaggtgctgggagcttggctttgttccctggtatccgcctggagacctgtgatgaaccactctggaacattcaggccaccatagagctgcagaccagccatgtggccaagggctgtgaccgtgacaactactcagagcgggcgctgcggaaactctgtggtgctgccactggggaggtggatctgttgcccatgcctggccccaatgccaactggacagcaggactctcggtgcactacagccaggacagcagcctgatctactggttcaatggcacccaggctgtgcaggtgcccctgggtggccccagtgggctgggctctgggccccaggacagcctcagtgaccacttcaccctgtccttctggatgaagcatggcgtaactcccaacaagggcaagaaggaagaggaaaccatcgtatgtaacactgtccagaatgaggacggcttctctcactactcgctgactgtccacggctgtaggattgccttcctctactggcccctgcttgagagtgcccgcccagtcaagttcctctggaagctggagcaggtctgtgatgatgagtggcaccactacgctctgaacctcgagttccccacagtcacactctataccgacggcatctccttcgaccctgccctcatccatgacaatggcctcatccacccaccccgaagggagcctgctctcatgattggggcctgctggactgaggagaagaacaaagagaaggaaaagggagacaacagtacagacaccacccaaggagaccctttgtcgatccaccactacttccatggctacctggctggtttcagcgtgcgctcaggtcgcctggagagccgcgaggtcatcgagtgcctctatgcatgtcgggaggggctggactatagggatttcgagagcctgggcaaaggcatgaaggtccacgtgaacccctcacagtccctgctcaccctggagggggatgatgtggagaccttcaaccatgccctgcagcatgtggcttacatgaacactctgcgctttgccacgcccggcgtcaggcccctgcgcctcaccactgctgtcaagtgcttcagcgaagagtcctgcgtctccatccctgaagtggagggctacgtggtcgtccttcagcctgacgccccccagatcctgctgagtggcactgctcattttgcccgcccagctgtggactttgagggaaccaacggcgtccctttgttccctgatcttcaaatcacctgctccatttctcaccaggtggaggccaaaaaggatgagagttggcagggcacagtgacagacacacgcatgtcggatgagattgtgcacaacctggatggctgtgaaatttctctggtgggggatgacctggatcccgagcgggaaagcctgctcctggacacaacctctctgcagcagcgggggctggagctcaccaacacatctgcctacctcactattgctggggtggagagcatcactgtgtatgaagagatcctgaggcaggctcgttatcggctgcgacacggagctgccctctacaccaggaagttccggctttcctgctcggaaatgaatggccgttactccagcaatgaattcatcgtggaggtcaatgtcctgcacagcatgaaccgggttgcccaccccagccacgtgctcagctcccagcagttcctgcaccgtggtcaccagcccccgcctgagatggctggacacagcctagccagctcccacagaaactccatgatacccagcgccgcaaccctcatcattgtggtgtgcgtgggcttcctggtgctcatggtcgtcctgggcctggtgcgcatccattcccttcaccgccgcgtctcaggggccggcgggcctccaggggcctccagtgaccccaaggacccagacctcttctgggatgactcagctctcaccatcattgtgaaccccatggagtcctaccagaatcggcagtcctgtgtgacgggggctgttgggggccagcaggaggatgaggacagcagtgactcggaggtggccgattcccccagcagcgacgagagacgcatcatcgagacccccccacaccgctactaaggcctacacctctccccacgcagagggggaattctgccctg

SEQ ID NO: 6 - protein, 956 aa, Homo sapiens
MTLLLLPLLLASLLASCSCNKANKHKPWIEAEYQGIVMENDNTVLLNPPLFALDKDAPLRYAGEICGFRLHGSGVPFEAVILDKATGEGLIRAKEPVDCEAQKEHTFTIQAYDCGEGPDGANTKKSHKATVHVRVNDVNEFAPVFVERLYRAAVTEGKLYDRILRVEAIDGDCSPQYSQICYYEILTPNTPFLIDNDGNIENTEKLQYSGERLYKFTVTAYDCGKKRAADDAEVEIQVKPTCKPSWQGWNKRIEYAPGAGSLALFPGIRLETCDEPLWNIQATIELQTSHVAKGCDRDNYSERALRKLCGAATGEVDLLPMPGPNANWTAGLSVHYSQDSSLIYWFNGTQAVQVPLGGPSGLGSGPQDSLSDHFTLSFWMKHGVTPNKGKKEEETIVCNTVQNEDGFSHYSLTVHGCRIAFLYWPLLESARPVKFLWKLEQVCDDEWHHYALNLEFPTVTLYTDGISFDPALIHDNGLIHPPRREPALMIGACWTEEKNKEKEKGDNSTDTTQGDPLSIHHYFHGYLAGFSVRSGRLESREVIECLYACREGLDYRDFESLGKGMKVHVNPSQSLLTLEGDDVETFNHALQHVAYMNTLRFATPGVRPLRLTTAVKCFSEESCVSIPEVEGYVVVLQPDAPQILLSGTAHFARPAVDFEGTNGVPLFPDLQITCSISHQVEAKKDESWQGTVTDTRMSDEIVHNLDGCEISLVGDDLDPERESLLLDTTSLQQRGLELTNTSAYLTIAGVESITVYEEILRQARYRLRHGAALYTRKFRLSCSEMNGRYSSNEFIVEVNVLHSMNRVAHPSHVLSSQQFLHRGHQPPPEMAGHSLASSHRNSMIPSAATLIIVVCVGFLVLMVVLGLVRIHSLHRRVSGAGGPPGASSDPKDPDLFWDDSALTIIVNPMESYQNRQSCVTGAVGGQQEDEDSSDSEVADSPSSDERRIIETPPHRY

SEQ ID NO: 7 - protein, 14 aa, Gallus gallus
ARVNKHKPWIETTY

SEQ ID NO: 8 - protein, 24 aa, Gallus gallus
HKPWIETTYHGIVTENDNTVLLDP

SEQ ID NO: 9 - protein, 6 aa, Gallus gallus
VEAVDA

SEQ ID NO: 10 - protein, 17 aa, Gallus gallus
IEYEPGTGSLALFPSMR

SEQ ID NO: 11 - protein, 7 aa, Gallus gallus
IPDGVVT

SEQ ID NO: 12 - protein, 9 aa, Gallus gallus
TYKPAEFHW

SEQ ID NO: 13 - protein, 11 aa, Gallus gallus
EGLDLQIADGV

SEQ ID NO: 14 - protein, 28 aa, Gallus gallus
GIEMSSSNLGMIITGVDTMASYEEVLHL

SEQ ID NO: 15 - DNA, 23 bp, Artificial Sequence
  Description of Artificial Sequence Primer
gtaaamaagcayaagccatggat

SEQ ID NO: 16 - DNA, 25 bp, Artificial Sequence
  Description of Artificial Sequence primer
caggaathgtaacagagaatgataa

SEQ ID NO: 17 - DNA, 23 bp, Artificial Sequence
  Description of Artificial Sequence primer
ccagtaccaggctcatactcdat

SEQ ID NO: 18 - DNA, 26 bp, Artificial Sequence
  Description of Artificial Sequence primer
gtatcaacaccatadatdatcatacc

SEQ ID NO: 19 - DNA, 23 bp, Artificial Sequence
  Description of Artificial Sequence primer
acaccatcagcdatctgaaaatc

SEQ ID NO: 20 - DNA, 26 bp, Artificial Sequence
  Description of Artificial Sequence primer
gcatcaaactcagcctccttataaaa

SEQ ID NO: 21 - DNA, 31 bp, Artificial Sequence
  Description of Artificial Sequence primer
ggggaacagaagagctgcacatcagcgaacg

SEQ ID NO: 22 - DNA, 29 bp, Artificial Sequence
  Description of Artificial Sequence primer
ccccctcgagttagtagctgagtgtggag

SEQ ID NO: 23 - DNA, 39 bp, Artificial Sequence
  Description of Artificial Sequence primer
gggccatggctcgtgttaacaagcataagccctggattg

SEQ ID NO: 24 - DNA, 47 bp, Artificial Sequence
  Description of Artificial Sequence primer
cccaagcttagtggtggtggtgatggtgtggttcatcacatgtgtcc

SEQ ID NO: 25 - protein, 268 aa, Gallus gallus
ARVNKHKPWIETTYHGIVTENDNTVLLDPPLIALDKDAPLRFAESFEVTVTKEGEICGFLKIHGQNVPFEAVVVDKSTGEGIIRSKEKLDCELQKDYTFTIQAYDCGKGPDGANAKKSHKATVHIQVNDVNEYSPVFKEKSYKATVIEGKRYDNILKVEAVDADCSPQFSQICNYEIVTPDVPFAIDKDGYIKNTEKLSYGKEHQYKLTVTAYDCGKKRAAEDVLVKISIKPTCKPGWQGWSKRIEYEPGTGSLALFPSMRLETCDEP

SEQ ID NO: 26 - DNA, 29 bp, Artificial Sequence
  Description of Artificial Sequenceprimer
gggccatgatacgctacagaaactggcac

SEQ ID NO: 27 - DNA, 49 bp, Artificial Sequence
  Description of Artificial Sequenceprimer
cccaagcttagtggtggtggtgatggtgagtggctgtacttggaacaac

SEQ ID NO: 28 - protein, 87 aa, Gallus gallus
IRYRNWHTVSLFDRKFKLVCSELNGRYVSNEFKVEVNVIHTANPIEHANHIAAQPQFVHPVHHTFVDLSGHNLANPHPFSVVPSTAT

SEQ ID NO: 29 - protein, 89 aa, Gallus gallus
LIRYRNWHTVSLFDRKFKLVCSELNGRYVSNEFKVEVNVIHTANPIEHANHIAAQPQFVHPVHHTFVDLSGHNLANPHPFSVVPSTATV

SEQ ID NO: 30 - protein, 89 aa, Gallus gallus
LLRYRNWHARSLLDRKFKLICSELNGRYISNEFKVEVNVIHTANPMEHANHMAAQPQFVHPEHRSFVDLSGHNLANPHPFAVVPSTATV

SEQ ID NO: 31 - DNA, 22 bp, Artificial Sequence
  Description of Artificial Sequence primer
ctcctctggcatcattgacctc

SEQ ID NO: 32 - DNA, 22 bp, Artificial Sequence
  Description of Artificial Sequence primer
catttcttcctcggcttcttcc

SEQ ID NO: 33 - DNA, 17 bp, Artificial Sequence
  Description of Artificial Sequence Primer
tgctgcgaggatgctgc

SEQ ID NO: 34 - DNA, 17 bp, Artificial Sequence
  Description of Artificial Sequence Primer
atgatgccagaggaggc

SEQ ID NO: 35 - protein, 85 aa, Homo sapiens
HIRYRNWRPASLEARRFRIKCSELNGRYTSNEFNLEVSILHEDQVSDKEHVNHLIVQPPFLQSVHHPESRSSIQHSSVVPSIATV

SEQ ID NO: 36 - DNA, 17 bp, Artificial Sequence
  Description of Artificial Sequence  primer
ctgcagtagcggggttg

SEQ ID NO: 37 - DNA, 21 bp, Artificial Sequence
  Description of Artificial Sequence primer
tggagtgtctgtttcaccagg

SEQ ID NO: 38 - protein, 87 aa, Homo sapiens
ILRQARYRLRHGAALYTRKFRLSCSEMNGRYSSNEFIVEVNVLHSMNRVAHPSHVLSSQQFLHRGHQPPPEMAGHSLASSHRNSMIP

SEQ ID NO: 39 - DNA, 26 bp, Artificial Sequence
  Description of Artificial Sequence  PCR Primer
cgggatcccgcatccgggccgcacat

SEQ ID NO: 40 - DNA, 28 bp, Artificial Sequence
  Description of Artificial Sequence PCR Primer
gggaattcctcagtagctgagggtggag

SEQ ID NO: 41 - DNA, 30 bp, Artificial Sequence
  Description of Artificial Sequence PCR Primer
ccggaattcccttctgatggggaccatcag

SEQ ID NO: 42 - DNA, 30 bp, Artificial Sequence
  Description of Artificial Sequence PCR Primer
ccgctcgagtcagtcttcccactcatcatc

(19) United States
(12) Patent Application Publication
(10) Pub. No.: US 2004/0019919 A1
(43) Pub. Date: Jan. 29, 2004

(54) Calcium binding proteins

(76) Inventors: Peter Sonderegger, (US); Gustav Hintsch, Z?uuml;rich (CH); Jochen Kinter, Z?uuml;rich (CH); Virginija Meskenaite, Z?uuml;rich (CH); Sabine Schrimpf, Z?uuml;rich (CH); Lorenz Vogt, Wetzikon (CH); Andreas Zurlinden, Zuuml;rich (CH)

Correspondence Address: Marshall Gerstein & Borun, Sears Tower, Suite 6300, 233 S Wacker Drive, Chicago, IL, 60606-6357, US

(21) Appl. No.: 10/380,705

(22) PCT Filed: Sep. 13, 2001
(86) PCT No.: PCT/IB01/01662

(30) Foreign Application Priority Data
Sep. 14, 2000 (EP) 00810830.0

(51) Int. Cl. A01K 67/027; C07H 21/04; A61K 38/17; C07K 14/47

(52) U.S. Cl. 800/14; 514/44; 435/69.1; 435/320.1; 435/325; 530/350; 536/23.2; 514/12

(57) ABSTRACT

There is described a novel class of calcium binding proteins of the nervous system, in particular calsyntenin-1-3. Calsyntenin proteins are valuable agents in the treatment of disorders of the nervous system, in particular the central nervous system. They are very useful for the development of drugs for the treatment of disorders of the nervous system.

### CROSS REFERENCE TO RELATED APPLICATIONS

This application claims the priority of European patent application 00810830.0, filed Sep. 14, 2000, the disclosure of which is incorporated herein by reference in its entirety.

### TECHNICAL FIELD

The present invention concerns a novel class of calcium binding proteins predominantly expressed in the nervous system.

### BACKGROUND ART

Nervous system related disorders, in particular central nervous system related disorders, are getting greater importance, be it due to the enhanced average age of the people, be it due to enhanced numbers of injured people due to the enhanced occurrence of potential dangers, be it due to enhanced occurrence of stress-related and other psychic disorders.

There is a great interest in obtaining more knowledge about the nervous system and disorders involving the nervous system, such as psychic disorders, such as pain development, regeneration of injured nerves etc., and in particular about healing such disorders or injuries, or at least ameliorating the state of a patient suffering from such disorders or injuries, and there is a great need for pharmaceutical and diagnostic preparation in said field. One approach to learn more about nervous system related disorders is the identification and characterisation of proteins involved in biochemical pathways of the nervous system. Calcium plays an important role in signalling pathways of the nervous system. Although a lot of DNA sequences of nervous system active proteins have been published, still a lot of such proteins have not yet been detected. Furthermore, also for most of the known sequences, their activity is still unknown.

For the above mentioned reasons there is a great interest to identify and characterise proteins expressed in the nervous system and playing a role in Calcium signalling or storage.

### DISCLOSURE OF THE INVENTION

In nervous system derived cDNA libraries, recently the DNA sequence encoding a protein with so far unknown activity has been published. It has now been found in the scope of the present invention that said protein plays an important role in the calcium signalling pathway and is only one member of a whole class of compounds with similar activity. Said nervous system active protein that in the scope of the present invention has been denominated calsyntenin-1, has been found to comprise a single-pass transmembrane segment with a large extracellular segment and a small (approximately 100 amino acids) cytoplasmic segment highly enriched in acidic amino acid residues.

Nothing has been known prior to the present invention about the cellular pattern of calsyntenin gene expression, the cellular and subcellular localization and the functional role of the calsyntenin proteins, and calsyntenin-related disorders.

Hence it is an object of the present invention to provide an isolated polypeptide for the use as a pharmaceutical comprising an amino acid sequence at least 50% identical to a sequence selected from the group consisting of:

a) a full length amino acid sequence selected from the group consisting of Seq. Id. No. 2 (Calsyntenin-1), Seq. Id. No. 4 (Calsyntenin-2) and Seq. Id. No. 6 (Calsyntenin-3),

b) a polypeptide comprising at least one, preferably two, most preferably three of amino acid sequences selected from the group consisting of: sequence of residues from about 46 to about 165, sequence of residues 166 to about 257, sequence of residues from about 774 to about 861 and sequence of residues from about 881 to about 981 of Seq. Id. No. 2,

c) a polypeptide comprising at least one, preferably two, most preferably three of amino acid sequences selected from the group consisting of: sequence of residues from about 66 to about 158, sequence of residues from about 182 to about 259, sequence of residues from about 751 to about 834 and sequence of residues from about 854 to about 955 of Seq. Id. No. 4,

d) a polypeptide comprising at least one, preferably two, most preferably three of amino acid sequences selected from the group consisting of: sequence of residues from about 51 to about 142, sequence of residues from about 167 to about 244, sequence of residues from about 759 to about 845 and sequence of residues from about 869 to about 956 of Seq Id. No. 6,

e) a polypeptide comprising at least one, preferably two, most preferably three of amino acid sequences selected from the group consisting of: sequence of residues from about 881 to about 981 of Seq. Id. No. 2, sequence of residues from about 854 to about 955 of Seq. Id. No. 4 and sequence of residues from about 869 to about 956 of Seq Id. No. 6,

and having calcium binding activity and/or having the capacity to bind to the Arp2/3 complex.

Polypeptides of the above defined group which comprise a ligand binding function are of particular interest, especially polypeptides comprising amino acid residues 46 to 165, residues from about 166 to about 257 of Seq.Id. No. 2; especially polypeptides comprising amino acid residues from about 66 to about 158, residues from about 182 to about 259 of Seq. Id. No. 4; especially polypeptides comprising amino acid residues from about 51 to 142, residues from about 167 to about 244 of Seq. Id. No.6.

Polypeptides as defined above comprising a proteolytic cleavage site are preferred, especially a polypeptide comprising amino acid residues from about 774 to about 861 of Seq. Id. No. 2

especially a polypeptide comprising amino acid residues form about 751 to about 834 of Seq.Id. No. 4,

especially a polypeptide comprising amino acid residues from about 759 to 845 of Seq. Id. No. 6.

Polypeptides as defined above comprising a calcium binding domain of Calsyntenin-1 and/or Calsyntenin-2 and/or Calsyntenin-3 are preferred i.e. polypeptides comprising amino acid residues from about 881 to about 981 of Seq. Id. No. 2 and/or polypeptides comprising amino acid residues from about 854 to about 955 of Seq. Id. No. 4 and/or polypeptides comprising amino acid residues from about 869 to about 956 of Seq. Id. No. 6.

Preferred are polypeptide sequences that are at least 60% identical and more preferably more then 70% identical to an amino acid sequence selected from the above defined group.

Another object of the present invention is an isolated polypeptide for the use as a pharmaceutical comprising an amino acid sequence selected from sequences comprising a stretch of at least 100 amino acids with a minimal identity percentage of 50%, preferably 55% and more preferably 60% to an amino acid sequence selected from the group consisting of Seq. Id. No. 2, Seq. Id. No. 4 and Seq. Id. No. 6 and said sequences having calcium binding activity and/or the capacity to bind to the Arp2/3 complex.

The polypeptide of the present invention is preferably a transmembrane protein which is expressed predominantly in cells of the nervous system, and which is more preferably expressed in neurons.

The polypeptide of the present invention is preferably localised in a postsynaptic membrane of synapses, more preferably localized in a membrane of a spine apparatus of spine synapses and/or in a membrane of subsynaptic endoplasmatic reticulum of shaft synapses. Of particular interest are proteins having their major calcium-binding domain in the cytoplasmic compartment. Preferred are polypeptides which are expressed in tumors and other preferred polypeptides have at least one binding site for the Arp2/3 complex. Said Arp2/3 binding site is a conserved acidic amino acid sequence motive comprising a conserved tryptophan and encompasses but is not limited to e.g. the sequence motives MDWDDS and LEWDDS (amino acid sequence given in single letter code).

The polypeptides of the present invention or fragments thereof which have a minimal length of about 50 amino acids are suitable for the use as a tool for the development of a pharmaceutical.

Another object of the present invention is an isolated nucleotide sequence or a partial sequence thereof encoding a polypeptide of the present invention for the use as pharmaceutical.

Another object of the present invention is an isolated nucleotide sequence encoding a polypeptide of the present invention or a fragment thereof which has, due to at least one point mutation, insertion or deletion lost its function.

A further object of the present invention is an isolated nucleotide sequence encoding a polypeptide of the present invention or a fragment thereof, respectively which has, due to at least one point mutation, insertion or deletion lost its function for the use as a diagnostic tool. Such sequences, usually have not more than 25 differences to the active segment within the Calcium binding intracellular region and/or the extracellular segment comprising the protease recognition site.

A mutated region or a region flanking the mutated region of the nucleotide sequence encoding a polypeptide of the present invention is e.g. useful for the design of primers or nucleotide probes that can be used in a diagnostic test to detect mutated DNA isolated from e.g. human tissue. Such test is e.g. suitable to predict whether cells are likely to undergo transformation leading to cancer development. The terms primer or nucleotide probe as used herein include oligonucleotide sequences comprised of ten or more deoxyribonucleotides or ribonucleotides.

DNA sequences of the present invention shall be understood to also include splice variants and DNA sequences that hybridize under stringent conditions to the sequences selected from the group consisting of Seq. Id. No. 1 (Calsyntenin-1), Seq. Id. No. 3 (Calsyntenin-2) and Seq. Id. No. 5 (Calsyntenin-3). Under stringent conditions hybridizing sequences in general are sequences with at least about 80% identity, preferably about 90% identity and most preferred 98% identity. Said sequences comprise sequences encoding amino acid sequences having calcium binding activity as well as such sequences that encode amino acid sequences without calcium binding activity, in particular such sequences that for small defects have lost said activity. Small defect usually means a point mutation, an insertion or a deletion in said sequences. Sequences of the present invention also comprise sequences encoding amino acid sequences spanning over the proteolytic cleavage site(s) in the extracellular segment of the coded protein as well as such sequences that encode amino acid sequences without proteolytic cleavage site(s), in particular such sequences that for small defects have lost said cleavage site(s). The sequences of the present invention also comprise sequences encoding the amino acid sequences of the proteolytically released fragments, as well as such sequences that encode amino acid sequences with mutations in the proteolytically released fragment. The term allele as used herein is intended to include sequences that differ by one or more nucleotide substitutions, additions or deletions, usually at most 20 differences in activity providing regions.

Another object of the present invention is the use of the polypeptides or the nucleotide sequences of the present invention or fragments thereof as a tool for the development of pharmaceuticals and as a tool for the screening of pharmaceutical agents.

The present invention furthermore concerns pharmaceutical compositions, that comprise such a DNA sequence and/or a polypeptide or a fragment thereof as defined above.

A pharmaceutical composition of the present invention can also comprise as at least one active substance (ingredient) a protein as defined above.

A pharmaceutical composition can furthermore comprise at least one further active compound, e.g. a compound that increases or reduces the calcium binding activity of said above defined protein, or that increases or decreases the amount of such a protein at its place of action in the body, or that prolongs or shortens the time of presence of such a protein at its place of action in the body.

The present invention furthermore encompasses a pharmaceutical composition that comprises as an at least one active compound a substance which enhances or inhibits the transcription of a mRNA derived from a DNA as defined above, or in that it enhances or inhibits the translation of such a DNA.

The present invention concerns as well a pharmaceutical composition, that comprises as an at least one active compound a compound that reduces or increases the calcium binding activity of a protein as defined above, or that increases or decreases the amount of such a protein at its place of action in the body, or that shortens or prolongs the time of presence of such a protein at its place of action in the body.

Another object of the present invention are proteins having a sequence as specified in Seq. Id. No. 4 and homologues of said sequence comprising proteins which have at least 60% identity with said sequences.

Another object of the present invention are proteins having a sequence as specified in Seq. Id. No. 6 and homologous of said sequence comprising proteins which have at least 98.5% identity with said sequence.

Another object of the present invention are nucleotide sequences which encode a protein as specified in Seq. Id. No. 4 or Seq. Id. No. 6. The coding sequence of the nucleotide sequence comprises all sequences encoding the amino acid sequence of Seq. Id. No. 4 or Seq. Id. No. 6 or homologues thereof. Also included are partial sequences of the nucleotide sequences described above. For instance, the calsyntenin encoding sequence preferably has a sequence at least 70% similarity to the nucleotide sequence encoding the amino acid sequence of Seq. Id. No. 4 or Seq. Id. No. 6.

The DNA sequences and/or proteins defined above are suitable for the use in screening assays and/or the treatment of disorders, preferably nervous system disorders, more preferably of the central nervous system, most preferably the brain e.g. due to lack of calcium binding activity, or due to excessive calcium binding activity or due to perturbed processing of intracellular calcium signals and in particular in order to prevent, ameliorate or cure disorders of the nervous system caused due to lack of cleavage or miscleavage or excessive cleavage of a protein of the present invention induced by at least one protease, in particular proteases selected from the group consisting of tissue-type plasminogen activator, abbreviated as tPA, urokinase-type plasminogen activator, abbreviated as uPA, or plasmin, or neurotrypsin or nuroserpin. Said disorders due to perturbed processing of intracellular calcium signals are preferably caused by perturbed processing of extracellular signals that regulate the cellular motility processes by means of regulating the activity of the Arp2/3 complex. Thus, a method for treating such diseases by use of a protein or a nucleotide sequence of the present invention is also encompassed.

Most preferably the present invention concerns DNA sequences or proteins for the minimization of the tissue destruction in stroke.

By a preparation comprising such DNA sequences or proteins, the minimization of the tissue destruction in stroke including brain infarction and ischemia, intracerebral hemorrhage, and subarrachnoid hemorrhage, as for example by exerting a protecting effect on the cells of the so-called penumbra zone surrounding the necrotic tissue, can be obtained.

Other disorders where an effective substance or preparation of this invention can be used, be it as pharmaceutical, be it as diagnostic agent, include as a suitable selection

the treatment of tissue destruction in traumatic brain injury, as for example by exerting a protective effect on the cells of the so-called penumbra zone surrounding the necrotic tissue,

the prevention, amelioration or cure of negative effects caused by neurodegenerative diseases, or neuroinflammatory diseases, as for example multiple sclerosis,

the reduction or prevention of negative effects on brain tissue caused by epileptic seizures,

the rescue of endangered neurons, as for example neurons endangered by hypoxia and ischemia, excitotoxicity, neuroinflammatory diseases and processes, epileptic seizures, and cancerous neoformations,

the axonal regeneration and/or restoration of synaptic integrity and functions,

the prevention, amelioration, or cure of retinal disorders, as for example retinal degeneration and retinal neoangiogenesis,

the cell death of cells of the nervous system, in particular a cell death in connection with damages of the nervous tissue, for example infarct of the brain and ischemic stroke, or hemorrhage of the brain, or trauma of the brain, and/or a cell death in connection with damages of the nervous tissue, which occur due to lack of oxygen or glucose or due to intoxication, and/or a cell death in connection with epileptic seizures, and/or a cell death in connection with neurodegenerative diseases and inherited genetic deficiencies of the nervous system,

the regeneration of injured, damaged, under-developed, or maldeveloped brain tissue and/or nervous tissue,

the reorganization of the brain or nervous areas that have remained intact after brain and/or nerve injuries or after the destruction or damage of brain areas,

the prevention, amelioration, or cure of pathological pain syndromes,

the amelioration or cure of disorders in the field of disorders of the psychic wellness, or the psychosomatic state of health, as for example nervosity or “inner unrest”, disorders in the field of the emotional functions, as for example states of anxiety,

the prevention, amelioration or cure of psychiatric disorders, in particular psychiatric disorders in the field of schizophrenia and schizophrenia-like disorders, including chronic schizophrenia, chronic schizo-affective disorders, unspecific disorders, including acute and chronic schizophrenia of various symptomatologies, as for example severe, non-remitting “Kraepelinic” schizophrenia, or as for example the DSM-III-R-prototype of the schizophrenia-like disorders, including episodic schizophrenic disorders, including delusionic schizophrenia-like disorders, including schizophrenia-like personality disorders, as for example schizophrenia-like personality disorders with mild symptomatics, including schizotypic personality disorders, including the latent forms of schizophrenic or schizophrenia-like disorders, including non-organic psychotic disorders, and/or in the field of the endogenic depressions or in the field of manic or manic-depressive disorders,

the treatment of tumors such as prevention or reduction of the growth, the expansion, the infiltration and the metastasis of primary and metastatic tumors, inhibition of the formation of new blood vessels or neoangiogenesis, in particular the treatment of brain tumors or tumors of the retina. Said tumors preferably involve in their growth, expansion, infiltration, metastasis and promotion of blood vessels or neoangiogenesis an enhanced activity of the Arp2/3 complex. Said enhanced activity of the Arp2/3 complex is preferably mediated by an abnormal or excessive or reduced regulatory function of one of the proteins of the present invention.

Said tumors preferably involve in their growth, expansion, infiltration, metastasis and promotion of blood vessels or neoangiogenesis at least one protease functionally connected with a polypeptide of the present invention. Said protease is preferably a member of one of the following families:

Serine Protease family such as tissue-type plasminogen activator (tPA), urokinase-type plasminogen activator (uPA), plasmin, thrombin, neurotrypsin, neuropsin, elastases, cathepsin G,

Matrix Metalloproteinases family such as collagenases, gelatinases, stromelysins, matrylisins,

Cystein Proteases family such as cathepsin B and cathepsin D.

The present invention also concerns the amelioration of the learning and memory functions in healthy persons, as well as in persons with reduced learning and memory functions.

In one additional aspect, the present invention concerns a method for the production of proteins as defined above, that is characterized in that suitable host procaryotic and eucaryotic cells, in particular mammalian cells, are transfected with a DNA sequence as defined above in a vector ensuring the expression of said DNA sequence, and in that said transfected cells are cultured under suitable conditions allowing expression of said protein.

In another object the present invention relates to a synthetic or chemical method for the production of polypeptides, peptides or nucleic acid sequences representing at least part of the sequences of the present invention and having the ability to mimic or to block, respectively, the biological activity or calsyntenin, in particular the calcium binding activity.

The DNA sequences and/or the proteins defined above can furthermore be used as means for the screening of drugs against calsyntenin protein involving disorders, but also active ingredients such as transcription enhancers or reducers and translation enhancers or reducers and activity enhancers or reducers.

Another object of the present invention is a protease or proteases cleaving the proteins of the present invention in their extracellular segment.

Furthermore the present invention relates to cell extracts comprising a protease which cleaves a polypeptide of the present invention. The protease can have endogenous origin or can be the product of a heterologous expression construct transformed or transfected into said cells.

Another object of the present invention is a method for the identification of a compound or an agent which modulates the activity of said proteases. Said method comprises contacting cells producing an active protease with a test compound and measuring changes in protease activity. In a preferred embodiment said cells are mammalian cells and the protease is expressed from a heterologous gene construct.

Furthermore, the present invention also comprises the use of a sequence as defined above as a means to produce antigens or as antigen for the production of antibodies.

Such antibodies can e.g. be antibodies that inhibit or promote the calcium binding function or antibodies that inhibit or promote the proteolytic cleavage of a protein as defined above or antibodies that can be used for immunohistochemical studies or diagnostic assays.

The present invention also regards transgenic animals comprising an exogenous DNA sequence as defined above. Such animals are suitable for the study of diseases and the test of active substances as defined above

Such animals are in particular non human mammals, such as mice.

Still a further aspect of the present invention concerns the use of a DNA sequence as defined above for the inactivation or the mutation of the corresponding endogenous gene by means of gene targeting techniques.

Such gene targeting techniques are for example the elimination of the gene in the mouse through homologous recombination or the replacement of the gene by a mutated form thereof.

A DNA sequence as defined above can, within the scope of the present invention, also be used for the preparation of a diagnostic preparation for the diagnosis of disorders due to defects or alterations in the genomic sequence comprising a coding sequence similar to but not identical with one of the coding sequences defined above.

The nucleic acid sequences of the present invention are of great interest in gene therapeutical applications in humans and in animals, as for example as parts of gene therapy vectors, such as biological and synthetic vectors, or as parts of artificial chromosomes.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1A shows dissociated neurons from the ventral halves of E6 chicken spinal cords seeded in the central compartment of a cell culture system,

FIG. 1B shows neurites of neurons from the ventral halves of E6 chicken spinal cords extending into the side compartment after 6 days of cultivation,

FIG. 1C shows a compartmental cell culture system, the cell culture surface is subdivided into three compartments by a Teflon divider,

FIG. 1D shows a fluorography of a two-dimensional SDS-PAGE gel of ³⁵S methionine labelled proteins released into the medium of both the central and the side compartments,

FIG. 1E shows a fluorography of a two-dimensional SDS-PAGE gel of ³⁵S methionine labelled proteins released into the medium of both the central and the side compartments,

FIG. 2 shows an alignment of amino acid sequences deduced from the single ORF in the human (hs), the mouse (mm) and the chicken (gg) cDNA of calsyntenin-1,

FIG. 3 shows a demonstration of the calcium binding capacity of the cytoplasmic moiety of calsyntenin-1,

FIG. 4A shows an expression pattern of calsyntenin-1 mRNA in a sagital section of an E18 mouse,

FIG. 4B shows a an expression pattern of calsyntenin-1 mRNA in a coronal section of an adult mouse brain,

FIG. 4C shows a Northern blot analysis of calsyntenin-1 mRNA in adult human tissues,

FIG. 4D shows a Western blot analysis of human and chicken calsyntenin-1 protein,

FIG. 4E shows a schematic drawing of the calsyntenin-1 protein,

FIG. 5A shows synaptic localisation of calsyntenin-1 by immunohistochemical staining in a section of the hippocampus of an adult rat,

FIG. 5B shows colocalization of calysntenin-1 with the synaptic marker synapthophysin,

FIG. 5C shows colocalization of calsyntenin-1 with the α2 subunit of the synaptic marker GABA_{A} receptor,

FIG. 5D shows colocalization of calsyntenin-1 with the GluR2 subunit of the AMPA receptor,

FIG. 6A shows an ultrastructural localization of calsyntenin-1 in the postsynaptic membrane of spine synapses,

FIG. 6B shows an ultrastructural localization of calsyntenin-1 in the postsynaptic membrane of spine synapses,

FIG. 6C an ultrastructural localization of calsyntenin-1 in the postsynaptic membrane of spine synapses,

FIG. 6D shows an ultrastructural localization of calsyntenin-1 in the postsynaptic membrane of spine synapses,

FIG. 6E an ultrastructural localization of calsyntenin-1 in the postsynaptic membrane of spine synapses,

FIG. 6F an ultrastructural localization of calsyntenin-1 in the postsynaptic membrane of spine synapses,

FIG. 6G an ultrastructural localization of calsyntenin-1 in the postsynaptic membrane of spine synapses,

FIG. 7 shows localisation of calsyntenin-1 in synaptosomes, but not in postsynaptic densities,

FIG. 8A shows ultrastructural localization of the transmembrane fragment of proteolytically cleaved calsyntenin-1 over the spine apparatus of spine synapses,

FIG. 8B shows ultrastructural localization of the transmembrane fragment of proteolytically cleaved calsyntenin-1 over the spine apparatus of spine synapses,

FIG. 8C shows ultrastructural localization of the transmembrane fragment of proteolytically cleaved calsyntenin-1 over the spine apparatus of spine synapses,

FIG. 8D shows ultrastructural localization of the transmembrane fragment of proteolytically cleaved calsyntenin-1 over the spine apparatus of spine synapses,

FIG. 8E shows ultrastructural localization of the transmembrane fragment of proteolytically cleaved calsyntenin-1 over the spine apparatus of spine synapses,

FIG. 9 shows a diagram of the protease dependent translocation of the postsynaptic Ca²⁺ binding of calsyntenin-1

FIG. 10A shows the localization of calsyntenin-1 in growth cones of cultured hippocampal neurons by indirect immunofluorescence staining,

FIG. 10B identifies one of the neuronal processes shown in FIG. 10A as an axon by indirect immunofluorescence staining with an antibody against the axonal marker protein Tau 1,

FIG. 10C shows the localization of calsyntenin-1 in growth cone of cultured hippocampal neurons by indirect immunofluorescence at higher mangification,

FIG. 11 shows the production of the full-length form of calsyntenin 1 and the N-terminal secreted fragment of cleaved calsyntenin by Western Blotting with antibodies against calsyntenin-1 (R63 and R71).

FIG. 12A shows the expression of calsyntenin-3 mRNA in different human organs by Northern blotting,

FIG. 12B shows the expression of calsyntenin-3 mRNA at cellular resolution in a horizontal section through a brain of an adult mouse and

FIG. 12C shows the expression of calsyntenin-3 mRNA at cellular resolution in a parasagittal section through a brain of an adult mouse.

FIG. 13 demonstrates the binding of the Arp2/3 complex to the cytoplasmic part of calsyntenin-1. Bovine brain extract was passed over a column containing bound GST-Cst_{C} fusion protein. The proteins collected in the flow-through fraction, in the wash fractions, and in the elution fractions were separated by SDS-PAGE and stained with silver staining (upper panel). The same protein fractions were also electrotransferred after SDS-PAGE to nitrocellulose paper and the presence of Arp2/3 complex was visualized by immunodetection using as a first antibody a commercially available antibody directed against the Arp3 subunit of the Arp2/3 complex (lower panel).

### MODES FOR CARRYING OUT THE INVENTION

Calsyntenin proteins are known to be expressed predominantly in the brain; the gene expression in the brain takes place nearly exclusively in the neurons.

As representatives of the novel class of calcium binding proteins the isolation and characterisation of calsyntenin-1, 2 and 3 are further described.

The coded peptide of calsyntenin-1 has a length of 1009 amino acids and contains a signal peptide of 28 amino acids. The mature protein is composed of 981 amino acids. The extracellular segment comprises 860 amino acids, the transmembrane segments has 21 amino acids, and the cytoplasmic segment has 99 amino acids.

A particularly interesting function of calsyntenin-1 is found in the segment forming the calcium binding cytoplasmic region.

Calsyntenin-1 has a cytoplasmic segment that is highly enriched in acidic amino acid residues and has the capacity of binding calcium ions. The cytoplasmic segment of the calsyntenin-1 functions as high-capacity, low-affinity calcium binding structure and it also contains high-affinity binding sites for calcium.

By this function, calsyntenin-1 retains calcium in the subsynaptic zone of excitatory and inhibitory synapses of the central and the peripheral nervous system. By this feature, calsyntenin-1 mediates the accumulation of calcium in the zone beneath the postsynaptic membrane and thereby modulate the calcium-mediated synaptic functions. By these functions, calsyntenin-1 maintains elevated calcium concentrations in the zone beneath the postsynaptic membrane and thereby, prolong the calcium signals in the zone beneath the postsynaptic membrane. An interesting aspect of calsyntenin-1 is its removal from the postsynaptic membrane by an endocytic process that follows the proteolytic cleavage within the extracellular segment. Therefore, calsyntenin-1 is subject to dynamic regulations by proteolytic cleavage by at least one synaptic protease.

In the scope of the present invention it could now be shown that calsyntenin-1 has also an in vivo activity making it a very useful tool for the diagnostic and therapy of protease involving disorders, in particular of the the nervous system, more particular of the central nervous system.

It is known that the expression of calsyntenin-1 during neural development starts at the beginning of the time range in which restructuration processes of synapses are observed, that in the adult nervous system, their expression is predominant in brain regions in which synapse plasticity occurs, and that a particularly high expression of calsyntenin-1 is found in the cerebral cortex, the hippocampus, and the amygdala of the mouse.

In the deeper structures of the brain, in the brain stem, and in the spinal cord of the adult mouse, a weaker expression of the calsyntenin-1 is found.

In the adult peripheral nervous system, calsyntenin-1 is also expressed, for example in the sensory ganglia neurons.

The gene expression pattern of calsyntenin-1 in the brain is extremely interesting, because these molecules are expressed in the adult nervous system predominantly in neurons of those regions that are thought to play an important role in learning and in memory functions.

The gene expression pattern of calsyntenin-1 in the cerebral cortex is extremely interesting, because a reduction of the cellular differentiation in the cerebral cortex has been found to be associated with schizophrenia.

Another prominent characteristic of calsyntenin-1 consists therein that it is secreted by neurons.

This fact—together with the function as a calcium binding protein and the expression pattern in the developing and adult brain—suggests that the calsyntenin-1 plays a role in the regulation of the calcium-mediated signals in brain areas which are involved in the processing and storage of learned behaviors, learned emotions, or memory contents.

Together with the recently found evidence for a role of extracellular proteases, in particular tissue-type plasminogen activator, in neural plasticity (see Frey et al., J. Neurosci. 16, pages 2057-2063, 1996; Huang et al., Proc. Natl. Acad. Sci. USA 93, pages 699-704, 1996), the expression pattern allows the assumption that the calcium binding activity of calsyntenin has a role in learning and memory operations, for example operations which are involved in the processing and storage of learned behaviors, learned emotions, or memory contents.

The fact that calsyntenin-1 is a substrate of proteases is particularly interesting, because for example the protease tissue-type plasminogen activator (tPA) has been found to play a role in the pathogenesis of neuronal cell damage or neuronal cell death in the context of excitotoxin-induced epileptic seizures (see Tsirka et al., Nature 377, pages 340-344, 1995).

The gene expression pattern of the calsyntenin-1 in the spinal cord and in the sensory ganglia is interesting, because these molecules are expressed in the adult nervous system in neurons of those brain regions that are thought to play a role in the processing of pain, as well as in the pathogenesis of pathological pain.

Calsyntenin-1 was found in connection with a study aimed at discovering proteins that are secreted from axons of neurons (see Stoeckli et al., Eur. J. Biochem. 180, pages 249-259, 1989). Their preparation has now been described in several papers that are herein comprised by reference (see Osterwalder et al., EMBO J. 15, pages 2944, 1996; Schrimpf et al. Human Neuroserpin (PI12): cDNA Cloning and Chromosomal Localization to 3q26, Genomics, Vol. 39, pages 1-8 (1997).

This procedure for the cloning can also be used for the isolation of homologous sequences of other species, such as mouse, rat, rabbit, guinea pig, cow, sheep, pig, primates, birds, zebra fish (Brachydanio rerio), Drosophila melanogaster, Caenorhabditis elegans etc. Such sequences are preferred for the veterinary use in order to avoid incompatibility reactions.

The coding nucleotide sequences obtained e.g. by the above described methods can be used for the production of proteins with the coded amino acid sequences as defined above.

The coding sequences of calsyntenin genes can also be used as starting sequence for the isolation of alleles and splice variants, or parts thereof, can be used as probes for the isolation of the genes corresponding to said sequences. For example the polymerase chain reaction and the nucleic acid hybridization technique can be used for this purpose.

The coding sequences of the calsyntenin genes can be used as starting sequences for so-called “site-directed mutagenesis”, in order to generate nucleotide sequences encoding proteins as defined above, in particular those shown in Seq. Id. No. 1, 3 and 5, or parts thereof, but whose nucleotide sequence is degenerated with respect to the sequences shown in Seq. Id. No. 1, 3 and 5 due to use of alternative codons. Such mutagenesis can be desired dependent of the host cells used for the expression of the protein of interest.

The coding sequences disclosed in this invention, can be used as starting sequences for the production of sequence variants exhibiting altered function by means of so-called site-directed mutagenesis. Such altered functions can e.g. provide for proteins with longer lifetime, i.e. slower degradation, enhanced activity etc.

The coding sequences can be used for the production of vectors for use in gene therapy and cell engineering.

The coding sequences can be used for the generation of transgenic animals overexpressing the coding and the coded sequences of the present invention.

The coding sequences can be used for the diagnostics of disorders in the gene corresponding to the sequences of the present invention.

The amino acid sequences coded by the above described nucleic acid sequences can be used as active substances, as antigens for the production of antibodies, and as targets for drug development.

In a further aspect, the present invention relates to the use of the polypeptides or the nucleotide sequences of the present invention or fragments thereof as a tool for the development of pharmaceuticals and as a tool for the screening of pharmaceutical agents, in particular screening assays for compounds binding a protein of the present invention. A preferred target sequence of the proteins of the present invention for the binding of such molecules is the extracellular part of the proteins of the present invention, in particular the domain/site showing blue sepharose binding capacity. Another target sequence for the binding of such molecules is the intracellular Arp2/3 complex binding domain, in particular a sequence comprising the motives MDWDDS.. and ..LEWDDS (amino acid sequence given in single letter code), of the proteins of the present invention.

Suitable in vitro assays for the identification of compounds which have an effect on the activity and/or stability and/or expression of the proteins of the present invention are for example in vitro assays employing biochemical or biophysical tests able to detect specific protein/ligand interactions and include e.g. MS/NMR as described in Moy et al. Anal. Chem. 73(3):571-81, 2001, high-throughput nuclear magnetic resonance-based screeening as described in Hajduk P J. J. Med. Chem. 42(13): 2315-7, 1999 or mass spectrometry-based strategies as described in Kaur S., J. Protein Chem., 16(5): 505-11, 1997 which are incorporated herein by reference in its entirety.

Said aspect of the present invention is based on the findings that calsyntenin-1, and most likely also the family members calsyntenin-2 and calsyntenin-3, are capable of binding the Arp2/3 complex. Binding of the cytosolic segment of calsyntenin-1 to the Arp2/3 complex indicates a role of the calsyntenin protein family in the regulation of cell motility. While studying the scientific literature dealing with interactions between cell surface proteins and the cellular cytoskeleton, we found that the cytoplasmic part of all proteins of the calsyntenin family (i.e. calsyntenin-1, calsyntenin-2, and calsyntenin-3) contains at least one intriguing motif of conserved amino acid sequences containing a conserved tryptophan. This motif is highly similar to conserved acidic amino acid motifs with a conserved tryptophan found in the Arp2/3-binding domain of most, if not all, of the currently known activators of the Arp2/3 complex. In the cytoplasmic segment of calsyntenin-1, this motif is found twice, once with the amino acid sequence ..MDWDDS.. and once with ..LEWDDS.. (amino acid sequence given in single letter code). The cytoplasmic sequence of calsyntenin-2 contains one ..MDWDDS.. and one .. LEWDDS... The cytoplasmic segment of calsyntenin-3 contains a single motif of this kind, namely ..LFWDDS... The Arp2/3 complex plays a central role in the regulation of actin-based cellular motility, by regulating actin filament growth and branching (for reviews see: Borisy and Svitkina, Curr. Opin. Cell Biol. 12: 104-112, 2000; Pantaloni et al., Science 292: 1502-1506; Higgs and Pollard, Annu. Rev. Biochem., 70: 649-676, 2001; and references therein). Arp2/3 activators containing a similar motif with conserved acidic amino acids and a tryptophan include human WASP (Abbreviation for: Wiscott Aldrich Syndrome Protein), the related human N-WASP, the human Scar/WAVE1 proteins, and cortactin, exhibiting the sequences ..DDEWDD, ..DDEWED and .. EVDWLE, and ..ADDWET.., respectively (for WASP, N-WASP, and Scar/WAVE1 see Higgs and Pollard, Annu. Rev. Biochem. 70: 649-676, 2001; for cortactin see Uruno et al., Nature Cell Biol. 3: 259-266, 2001). The importance of the conserved tryptophan and the adjacent acidic amino acids for Arp2/3 binding and Arp2/3 function in actin polymerization has been demonstrated by site-directed mutagenesis of cortactin (Uruno et al., Nature Cell Biol. 3: 259-266, 2001). Site directed mutagenesis of both the tryptophan and the two amino acid residues preceeding the tryptophan in the sequence ..ADDWET.. of cortactin resulted in the loss of Arp2/3 binding and Arp2/3-mediated actin polymerization. All these Arp2/3 activator proteins residene in the cytoplasm. They link intracellular signals derived from the interaction of transmembrane receptors with their extracellular regulators, such as growth factor, cytokines, etc., to activation of the Arp2/3 complex. A crucial intermediate step in the signaling cascade from activated transmembrane receptors to the activation of the Arp2/3 activators has been attributed to the small GTP-binding proteins of the Rho family (for a review: Takai et al., Physiol. Rev. 81:153-207, 2001). Activated Arp2/3 complex initiates the generation of new actin filaments and the branching of pre-existing actin filaments (for reviews see: Borisy and Svitkina, Curr. Opin. Cell Biol. 12: 104-112, 2000; Pantaloni et al., Science 292: 1502-1506; Higgs and Pollard, Annu. Rev. Biochem., 70: 649-676, 2001; and references therein). As a result of the enhanced cytoskeletal dynamics, the cells generate and/or retract plasma membrane protrusions, such as filopodia and lamellipodia (Borisy and Svitkina, Curr. Opin. Cell Biol. 12: 104-112, 2000). This enhanced activity translates into an enhanced exploratory activity of the growth cones, the growing tip of the axons extending from neurons as well as enhanced axon growth and pathfinding activity (Hu and Reichardt, Neuron 22, 419-422, 1999; Suter and Forscher, Curr. Opin. Neurobiol. 8: 106-116, 1998; Dickson, Curr. Opin. Neurobiol. 11: 103-110, 2001). In the dendritic spines of neurons of the central nervous system, the enhanced dynamics of actin filaments results in an increase in motility, which in turn may regulate the morphological shape and the electrical properties of the spine. As a consequence, the postsynaptic response to presynaptic signals may be altered (Segal et al., Trends Neurosci. 23: 53-57, 2000; Halpain, Trends Neurosci. 23: 141-146, 2000; Matus, Science 290: 754-758, 2000; Scott and Luo, Nature Neurosci. 4: 359-365, 2001). In non-neuronal cells, the intensification of actin filament dynamics induced via Arp2/3 activation results in an enhanced cell motility that is accompanied by enhanced formation of membrane protrusions, such as lamellipodia, and enhanced migratory activity (Holt and Koffer, Trends Cell Biol. 11: 38-47, 2001; Mullins, Curr. Opin. Cell Biol. 12: 91-96, 2000; Prokopenko et al., J. Cell Biol. 148: 843-848, 2000). A dysregulated signalling from the cell surface to the cytoskeleton changes the migratory activity of tumor cells that is linked to their enhanced capacity for invasive growth and metastasis (Radisky et al., Seminars Cancer Biol. 11:87-95, 2001; Kassis et al., Seminars Cancer Biol. 11:105-119, 2001; Condeelis et al., Seminars Cancer Biol. 11:119-128, 2001; Price and Collard, Seminars Cancer Biol. 11:167-173, 2001).

The present invention provides methods to evaluate the activity of a compound to selectively regulate synaptic calcium signals. The rationale of the screening approach presented here is based on the immunoelectron microscopic studies presented herein. In these studies we found that full-length calsyntenin-1 is almost exclusively located in and beneath the postsynaptic membrane, whereas the transmembrane fragments generated by proteolytic cleavage is translocated to the membranes of the so-called spine apparatus. The complete absence of full-length calsyntenin-1 from the spine apparatus indicates that only proteolytically cleaved calsyntenin-1 is internalized. Obviously, the proteolytic cleavage is a prerequisite for the internalization of the transmembrane segment of calsyntenin-1. As a result of the proteolytic cleavage of calsyntenin-1 and the subsequent internalization of its transmembrane fragment, the amount of calsyntenin-1 in the postsynaptic membranes is decreased. As a consequence, the regulatory influence of the cytoplasmic segment of calsyntenin-1 on synaptic calcium signaling is decreased.

Based on these characteristics, the proteolytic cleavage of calsyntenin-1 in its extracellular segment correlates with a reduction of the calsyntenin-1-mediated calcium-binding capacity beneath the postsynaptic membrane. Therefore, the extent of the proteolytic cleavage of calsyntenin-1 provides a correlate for the calsyntenin-1-mediated regulation of postsynaptic calcium signals. This link between proteolytic cleavage of calsyntenin-1 and the calsyntenin-1-mediated regulation of synaptic calcium signals can be exploited for the establishment of a relatively simple assay for testing a compound for its potential activity as a modulator of synaptic calcium signalling. This assay comprises contacting a calsyntenin protein expressing and synapse-forming neuronal cell culture or a synaptosomal or synaptoneurosomal preparation with a preselected amount of the compound in a suitable culture medium or buffer. After a suitable period of incubation, the progress of the proteolytic cleavage reaction of a full-length calsyntenin protein is assessed by measuring the decrease in the full-length form of calsyntenin and the increase in the two cleavage products. Measuring the degradation of a full-length calsyntenin protein and/or the generation of cleavage products of a calsyntenin protein by said neuronal cell culture or said synaptosomes or synaptoneurosomes, as compared to a control, will provide a measure for the efficiency of a compound in modulating endocytosis of a calsyntenin protein and, thus, the translocation of the calsyntenin-binding domain from the zone beneath the postsynaptic membrane of the spine apparatus and, thus, the modulation of postsynaptic calcium signals.

More specifically, the present invention provides a method of determining the ability of a compound to influence the cleavage of a calsyntenin protein in the extracellular moiety. A typical experiment consists in:

a) preparation of a synapse-forming neuronal cell culture (e.g. dissociated hippocampal culture from mouse or rat brain: Goslin et al., 1998, Cultering nerve cells, 2^{nd} Ed., MIT Press Cambridge, Mass.; or, alternatively, preparation of synaptosomes or synaptoneurosomes from brains of rodents (mouse or rat) or birds (chicken or pigeon) using established protocols (for synaptosomes: Phelan and Gordon-Weeks, 1997, Neurochemistry, A practical approach, 2^{nd} Ed.; for synaptoneurosomes: Hollingsworth et al., 1985, J. Neurosci. 5, 2240-2253)

b) addition of the compound to the culture medium of the synapse-forming neuronal cell culture or to the buffer containing the suspended synaptosomes or synaptoneurosomes.

c) separation of the cellular or synaptosomal or synaptoneurosomal proteins by sodiumdodecylsulfate polyacrylamide gel electrophoresis.

d) visualization of a full-length calsyntenin and fragments thereof by Western blot analysis.

e) measurement of the relative amounts of full-length and cleaved calsyntenin.

f) comparison of the relative amounts of cleaved and uncleaved calsyntenin with the relative amounts of cleaved and uncleaved calsyntenin obtained under the control condition.

The present invention provides simple in vitro systems for the screening of drug actions on synaptic calcium signalling, which will be useful for the development of drugs that selectively modulate synaptic calcium signal without producing side effects due to modulation of nonsynaptic calcium signals. Assays can be performed on living synapse-forming cultures of mammalian or avian neurons or on isolated mammalian or avian synapses (so-called synaptosomes or synaptoneurosomes), which can be cultivated or prepared, respectively, with relative ease. The assessment of the proteolytic cleavage of a calsyntenin by Western blot analysis is a relatively simple procedure as well. Thus, the assay is suited for high-throughput screening of a large number of compounds.

The invention also relates to methods for the identification of genes, termed “pathway genes”, which are associated with a calsyntenin gene product or with the biochemical pathways which extend therefrom. “Pathway gene”, as used herein, refers to a gene whose gene product exhibits the ability to interact with a calsyntenin gene product.

Any method suitable for detecting protein-protein interactions may be employed for identifying pathway gene products by identifying interactions between gene products and a calsyntenin gene product. Such known gene products may be an intracellular, a transmembranal, or an extracellular protein. Those gene products which interact with such known gene products represent pathway gene products and the genes which encode them represent pathway genes.

Among the traditional methods which may be employed are co-immunoprecipitation, crosslinking and co-purification through gradients or chromatographic columns. Utilizing procedures such as these allows for the identification of pathway gene products. Once identified, a pathway gene product may be used, in conjunction with standard techniques, to identify its corresponding pathway gene. For example, at least a portion of the amino acid sequence of the pathway gene product may be ascertained using techniques well known to those of skill in the art, such as via the Edman degradation technique (see, e.g., Creighton, 1983, Proteins: Structures and Molecular Principles, W. H. Freeman & Co., New York, pp.34-49). The amino acid sequence obtained may be used as a guide for the generation of oligonucleotide mixtures that can be used to screen for pathway gene sequences. Screening made be accomplished, for example by standard hybridization or PCR techniques. Techniques for the generation of oligonucleotide mixtures and screening are well-known. (See, e.g., Ausubel et al., eds., 1987-2000, Current Protocols in Molecular Biology, John Wiley & Sons, Inc. New York, and PCR Protocols: A Guide to Methods and Applications, 1990, Innis, M. et al., eds. Academic Press, Inc., New York).

Additionally, methods may be employed which result in the simultaneous identification of pathway genes which encode the protein interacting with a calsyntenin gene product. These methods include, for example, probing expression libraries with a labeled calsyntenin protein, using this protein in a manner similar to the well known technique of antibody probing of lambda gt11 libraries.

One such method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to a known protein, and the other consists of the activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the activator's binding sites. Either hybrid protein alone cannot activate transcription of the reporter gene: the DNA-binding domain hybrid because it does not provide activation function and the activation domain hybrid because it cannot localize to the activator's binding sites. Interaction of the two proteins reconstitutes the functional activator protein and results in expression of the reporter gene, which is detected by an assay for the reporter gene product.

The two-hybrid system or related methodology may be used to screen activation domain libraries for proteins that interact with a calsyntenin gene product, herein also called the known “bait” gene protein. Total genomic or cDNA sequences may be fused to the DNA encoding an activation domain. Such a library and a plasmid encoding a hybrid of the bait gene protein fused to the DNA-binding domain may be cotransformed into a yeast reporter strain, and the resulting transformants may be screened for those that express the reporter gene. These colonies may be purified and the library plasmids responsible for reporter gene expression may be isolated. DNA sequencing may then be used to identify the proteins encoded by the library plasmids.

For example, and not by way of limitation, the bait gene may be cloned into a vector such that it is translationally fused to the DNA encoding the DNA-binding domain of the GAL4 protein.

A cDNA library of the cell line from which proteins that interact with bait gene are to be detected can be made using methods routinely practiced in the art. According to the particular system described herein, for example, the cDNA fragments may be inserted into a vector such that they are translationally fused to the activation domain of GAL4. This library may be co-transformed along with the bait gene-GAL4 fusion plasmid into a yeast strain which contains a lacZ gene driven by a promoter which contains the GAL4 activation sequence. A cDNA encoded protein, fused to the GAL4 activation domain, that interacts with bait gene will reconstitute an active GAL4 protein and thereby drive expression of the lacZ gene. Colonies which express lacZ may be detected by their blue color in the presence of X-gal. The cDNA may then be purified from these strains, and used to produce and isolate the bait gene-interacting protein using techniques routinely practiced in the art.

Another method for discovering pathway genes is eucaryotic expression cloning. The expression-cloning method allows the isolation of a cDNA encoding a molecule that physically interacts with the protein of interest from a cDNA library contained in a eucaryotic expression vector. Over the past years it has emerged as a powerful method to identify the binding partners of many different proteins and other molecules (see Simmons, 1993, Cloning cell surface molecules by transient expression in mammalian cells, IRL Press at Oxford University Press, New York; Ausubel et al., 2000, Curr. Protocols in Molec. Biol). In contrast to the two-hybrid system technologies, it is the preferred technology to detect protein-protein interactions in the extracellular compartment. One version of this method is described here in detail for illustration only an not for limitation.

The expression cloning approach involves:

construction and/or purification of a probe, that will be used for screening

creation of a cDNA expression library from a suitable mRNA source

screening of the expression library and determination of the positive pools that express a ligand interacting with the probe

subcloning until a single clone bearing the cDNA of the ligand is found.

The screening of cDNA expression libraries in cultured mammalian cells is more laborious than the screening of a phage or plasmid library in bacterial cultures. It requires the amplification of the plasmid DNA of the cDNA library clones in bacteria, its isolation and the subsequent transfection into mammalian cells for expression. Yet, mammalian cells, such as COS cells, are ideal hosts for said purpose. These cells are able to synthesize long translation products correctly from the cDNA template and to carry out the folding of the protein and its postranslational modifications in the extracellular compartment correctly. One possibility to design the probe for the screening of a cDNA expression library is fusion of the protein of interest with alkaline phosphatase (Flanagan a Leder, 1990, Cell 63, 185-194). Alkaline phosphatase (AP) has an intrinsic enzyme activity that can be used to trace the fusion protein with high sensitivity. A wide variety of substrates for AP allows quantitative assays in solutions or in situ detection. Since antibodies against AP are available, the immunological detection of AP-fusion protein is also possible.

For the production of the AP-tagged fusion protein, eucaryotic expression-vectors, such as pcDNA3.1 (Invitrogen) are suitable. Because the interaction of a calsyntenin with its ligand may be perturbed, if the binding site is located near the region where the AP tag is fused, two constructs need to be generated for each fragment of a calsyntenin included in the screening program, one with the AP tag fused to the 3′-end and another with the AP tag fused to the 5′-end. The fusion proteins can be produced by transient expression in a suitable eucaryotic cell line, such as HEK 293T or COS. The well-expressed proteins are tested for example by Western blot analysis and quantified by an AP assay. As calsyntenin proteins are expressed in neurons throughout the brain, including the hippocampus, the probes may be tested for binding to cultures of dissociated hippocampal neurons. Testing of the probes with cultured neurons from other brain regions or with tissue slices is also possible.

To obtain a control for expression and binding experiments the sequence of secreted AP can be inserted in the same expression vector. Use of the endogenous secretion signal and the Kozak sequence of a calsyntenin or AP, together with the CMV promoter warrants the efficient translation of the fusion protein and its secretion into the culture medium.

The vector thus generated is transfected into the human embryonic kidney cell line, HEK293T, using the calcium phosphate transfection method or another transfection method, such as electroporation or lipofection. The conditioned medium is collected 3-4 days after transfection. The amount of the expressed protein is estimated by measuring the AP activity in the conditioned medium. A sample of conditioned medium is diluted in a buffer containing the soluble substrate of AP. The velocity of conversion of the substrate by AP is proportional to the activity of this enzyme. Since AP converts the substrate into a color product, it is possible to measure it spectrofotometrically. The expressed proteins are also tested by Western blot analysis using a polyclonal antibody against AP. Thus, the apparent molecular weight of the fusion protein may be determined as a control for its integrity.

Eucaryotic expression cloning requires a suitable RNA-source for generating a cDNA library. The mRNAs encoding the calsyntenin proteins are expressed predominantly in the neurons of the central nervous system, including the neurons of the hippocampus. Immunohistochemical staining of mouse brain sections confirmed the calsyntenin-1 expression in these brain regions also on the protein level. Therefore, it is plausible to assume that the putative ligand is expressed in the same regions where a calsyntenin protein is located. To test this hypothesis the dissociated hippocampal cell cultures were prepared and tested on binding of the probes. The binding assay is carried out according to the following protocol. The dissociated hippocampal neurons are shortly prefixed and incubated for 90 minutes with the buffered conditioned medium containing the appropriate probe. Subsequently the cells are washed and subjected to a short pre-fixation. Endogenous heat-sensitive AP is then inactivated by incubation of cells at 65° C. for 2 hours. The AP inserted into the fusion proteins is heat-stable and remains active after this step. At the end the cells are incubated with the staining buffer containing a substrate that can be converted by AP into a colored precipitate. Therefore, the cells that express a molecule that binds the probe are stained blue.

The cDNA for the generation of a expression library can be generated from mRNA obtained from the brain of an adult mouse, or rat, or human by a standard technique (see Ausubel et al., 2000). Eucaryotic expression vectors for the transfection of the library into COS cells include for example pCDM8 (Aruffo and Seed, 1987, Proc. Natl. Acad. Sci. USA 84, 8573-8577) or, more recently, pcDNA31 (Invitrogen). For the generation of the library, various protocols have been successfully used (see e.g. Simmons, 1993). Immediately after generation, a cDNA expression library can be divided into approximately 200 pools with complexity 1000-1500 colony-forming units (cfu) per pool. Each of the pools is plated out for example in triplicate. 500-1.000 cfu are grown on each plate. After 36 hours, when the colonies have reached to diameter of 2-3 mm, the bacteria are washed from the plates with the medium. A part of the bacterial suspension can be mixed with glycerol and stored frozen as a back-up for subsequent subpooling. The rest of the suspension can be used for the isolation of the plasmid DNA. For example COS cells are transfected with the plasmid DNA of individual cDNA library pools and after 48 hours, when the cDNA fragments are expressed; they are tested on the probe binding. The efficiency of the transfection and quality of the staining reaction was always controlled by transfection of the cells with cDNA of neuropilin-1 and staining of these control cells with its known binding partner semaphorin-III fused to AP. As a negative control mock transfected cells stained with both calsyntenin-AP and semaphorin-AP. All the cDNA library pools can be screened in triplicate.

All the positive pools can be subjected to the subpooling procedure. From the back-up of each positive pool 50 plates are plated, so that on each plate about 100 cfu are present. When the colonies become visible a replica is made. Both replica and original plate are incubated further till the colonies reach a diameter of about 2-3 mm. Then the bacteria are washed from the replica plates and the plasmid DNA is isolated. The original plates are stored at 4° C. for the next round of subpooling. The COS cells are then transfected with the isolated DNA and after 2 days tested with the same probe.

Once a pathway gene has been identified and isolated, it may be further characterized as, for example, discussed herein.

The proteins identified as products of pathway genes may be used to modulate gene expression of a calsyntenin, as defined herein. Aternatively, the proteins identified as products of pathway genes may be used to modulte the proteolytic cleaveage of a calsyntenin and the resulting internalization of the transmembrane fragement of a calsyntenin with its calcium-binding cytoplasmic domain. Alternatively, the proteins identified as products of pathway genes may be used to modulate the influence of the calcium-binding domain of a calsyntenin in synaptic calcium signaling. Pathway genes may themselves be targets for modulation to in turn modulate calsyntenin protein function.

The compounds identified in the screen will demonstrate the ability to selectively modulate the activity of a calsyntenin protein as a modulator of synaptic calcium signaling. These compounds include, but are not limited to, small organic molecules that regulate the proteolytic activity of the protease(s) that cleave(s) calsyntenin proteins in the extracellular part, molecules that bind the extracellular part of a calsyntenin protein and thereby modulate the susceptibility of a calsyntenin protein for proteolytic cleavage and/or internalization, molecules that bind to the transmembrane or cytoplasmic domain of a calsyntenin protein and modulate its affinity for calcium or its capacity of binding calcium, and molecules that bind to any part of a calsyntenin protein and modulate its interaction with macromolecular ligands, such as those defined herein as pathway proteins or pathway genes. These compounds also include, but are not limited to, nucleic acid encoding a calsyntenin protein and homologues, analogues, and deletions thereof, as well as antisense, ribozyme, triple helix, double-stranded RNA, antibody, and polypeptide molecules and small inorganic or organic molecules.

Any of the identified compounds can be administered to an animal host, including a human patient, by itself, or in pharmaceutical compositions where it is mixed with suitable carriers or excipient(s) at doses therapeutically effective to treat or ameliorate a variety of disorders, including those characterized by insufficient, aberrant, or excessive calsyntenin activity. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition.

A number of disorders may result from insufficient, aberrant, or excessive calsyntenin protein activity. In addition, several physiological states which may, from time to time be considered undesired, may also be associated with calsyntenin activity. By way of example, but not by way of limitation, such disorders and physiological states which may be treated with the compounds of the invention include but are not limited to psychiatric disorders such as schizophrenia or depression, neurologic disorders such as Alzheimer's disease, stroke, and acute head injury, acute or chronical headache, hypertension, and myocardial infarction.

Other options may include direct delivery of enzyme which has been produced and purified by genetic means using the cloned gene. Other isoforms may exist and may be cloned utilizing a calsyntenin sequence. The compounds of the invention may be designed or administered for tissue specificity. If the compound comprises a nucleic acid molecule, including those comprising an expression vector, it may be linked to a regulatory sequence which is specific for the target tissue, such as the brain, kidney, heart, etc. by methods which are known in the art including those set forth in Hart, 1994, Ann. Oncol., 5 Suppl 4: 59-65; Dahler et al., 1994, Gene, 145: 305-310; DiMaio et al., 1994, Surgery, 116:205-213; Weichselbaum et al., Cancer Res., 54:4266-4269; Harris et al., 1994, Cancer, 74 (Suppl. 3):1021-1025; Rettinger et al., Proc. Nat'l. Acad. Sci. USA, 91:1460-1464; and Xu et al, Exp. Hematol., 22:223-230; Brigham et al., 1994, Prog. Clin. Biol. Res., 388:361-365. The compounds of the invention may be targeted to specific sites by direct injection to those sites. Compounds designed for use in the central nervous system should be able to cross the blood brain barrier or be suitable for administration by localized injection. In addition, the compounds of the invention which remain within the vascular system may be useful in the treatment of vascular inflammation which might arise as a result of arteriosclerosis, balloon angioplasty, catheterization, myocardial infarction, vascular occlusion, and vascular surgery. Such compounds which remain within the bloodstream may be prepared by methods well known in the art including those described more fully in McIntire, 1994, Annals Biomed. Engineering, 22:2-13.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC50 (the dose where 50% of the cells show the desired effects) as determined in cell culture. Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). Dosage amount and interval may be adjusted individually to provide plasma levels of the active moiety which are suffficient to maintain the desired effects.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgement of the prescribing physician.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration,the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or′insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described-previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation. Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition. Suitable conditions indicated on the label may include treatment of a disease such as one characterized by insufficient, aberrant, or excessive calsyntenin-1 activity.

The just outlined uses of nucleic acid sequences and amino acid sequences as defined above has been shown in the scope of the present invention to be very suitable for protease involving disorders, in particular tPA involving diseases, and especially suitable for the treatment of stroke. For example in stroke treatment the calsyntenins or the calsyntenin derived proteins are suitable pharmaceuticals in acute treatment as well as in long-time treatment.

In an acute state, i.e. within the first few hours after a stroke, a presently preferred mode of application is the direct application of a high amount of a calsyntenin protein, preferably an intrathecal application, i.e. an injection directly into the cerebro-spinal fluids.

For the long term therapy of stroke, i.e. the restitution of damages, a preferred method is cell therapy.

For gene therapy and/or cell therapy a nucleic acid sequence coding for a calsyntenin protein (the expression a calsyntenin protein is considered as including alleles and mutants with protease inhibitor, at least tPA inhibitor activity) is introduced into a suitable vector allowing the expression of a calsyntenin gene in the addressed nerve cells or specific therapy cells. Such a vector suitable for gene therapy and allowing expression of the calsyntenin comprises the calsyntenin-1 encoding gene under the control of a nerve cell specific promoter.

For gene therapy suitable vectors are neurotrophic viruses that can be applied either directly or in transport cells.

Calsyntenin expressing cells can also be encapsulated so that they can be brought to the center of desired action by surgery treatment and with much reduced risk for incompatibility reactions. Such cells can be removed as soon as they are no longer needed or as soon as they have lost their activity and thus need replacement.

All the above described methods for the treatment of stroke are similarly applicable to other disorders induced by proteases, in particular tPA. Such disorders also comprise tumors such as those induced by tPA due to its effect on cell migration, but also tumors generally involving at least one protease in their growth, expansion, infiltration, metastasis and promotion of blood vessels or neoangiogenesis. Such proteases are preferably members of at least one of the following protease families:

Serine Protease family such as tissue-type plasminogen activator (tPA), urokinase-type plasminogen activator (uPA), plasmin, thrombin, elastases, cathepsin G, neuropsin, neurotrypsin

Matrix Metalloproteinases family such as collagenases, gelatinases, stromelysins, matrilysins,

Cystein Proteases family such as cathepsin B and cathepsin D.

Besides of the above further described treatments, the present invention also provides for very useful diagnostic tools. By PCR and hybridization methods, as already mentioned above, genetic defects in the calsyntenin encoding protein can be determined. Such determination helps for the diagnosis of disorders the symptoms of which are already noticeable as well as for the determination of persons or groups of persons, such as families, with enhanced risk to develop such a disorder.

It is of course also possible to produce by synthetic or chemical methods proteins, peptides or nucleic acid sequences representing at least part of the sequences defined above and having the ability to mimic or to block, respectively, the biological activity of calsyntenin, in particular the calcium binding activity.

Furthermore, the characterization and isolation of a deficient gene or a deficient protein encoded by such a gene provides efficient tools for screening possible drugs to improve the health of patients suffering from disorders due to such defects.

In particular for the search of further disorders and drugs also transgenic animals are of great value.

This and further aspects of the present invention are now further illustrated by the following examples. It has, however to be understood that they are not at all intended to reduce the scope of the present invention. They are of mere illustrative purpose.

### EXAMPLE 1

Screening for proteins released from the neurites of embryonic chicken spinal cord neurons identifies a 115 kD protein as a proteolytic fragment of calsyntenin-1.

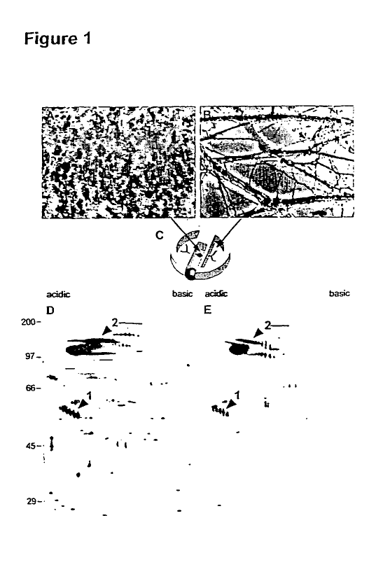

In a search for proteins released from neurites we cultivated dissociated spinal cord neurons in a compartmental cell culture system that provides separate access to neuronal cell bodies and neurites (FIG. 1C). The compartmental cell culture system was set up as described by Campenot (1979), Methods Enzymol, 58, 302-7. Dissociated cells from the ventral halves of spinal cords of E6 chicken embryos were cultivated in the center compartment of the compartmental culture system as described previously (Sonderegger et al. 1984, J.Cell. Biol. 98(1): 364-8). Six days after plating, when the side compartments had become densely populated by neurites (FIG. 1B), the newly synthesized proteins were metabolically labeled by adding fresh medium containing [³⁵S] methionine to the center compartment ( Stoeckli et al., 1989, Eur. J. Biochem. 180(2): 249-58). After 40 hours, the conditioned media of both the center and the side compartments were harvested and subjected to two-dimensional gel electrophoresis (O'Farrell, J Biol Chem. May 25, 1975;250(10): 4007-21.) followed by fluorographic detection (Bonner and Laskey, Bur J Biochem. Jul. 1, 1974;46(1):83-8.) of the newly synthesized proteins (FIGS. 1D and E). As shown in FIG. 1D, the supernatant of the center compartment contained a relatively large number of proteins, whereas only four strong protein spots were found in the side compartment (FIG. 1E). Because proteins diffusing from the center to the side compartment did not reach more than 10% of their concentration in the center compartment, we concluded that these four proteins had to be derived from the neurites of the side compartment (for a quantitative study with the same system see (Stoeckli et al., 1989, Eur. J. Biochem. 180(2): 249-58). One of them (FIG. 1E, arrow 1) was previously identified as neuroserpin, an axonally secreted serine protease inhibitor (Osterwalder et al., EMBO J. 1996; 15(12):2944-53.). Based on molecular weight and pI, three other proteins were unknown. The protein with an apparent molecular weight of 115 kD and a pI of 5.9 to 6.3 (arrow 2 in FIG. 1E) was isolated and characterized as reported in the following examples. Purification, amino acid sequencing, and cDNA cloning (see examples 2 and 3) revealed that the 115 kD protein released from the neurites of embryonic chicken spinal cord neurons is a proteolytic fragment of a transmembrane protein. Because of its synaptic localization (see examples 10 and 11) and its capacity to bind calcium with its cytoplasmic domain (example 6), we termed it calsyntenin-1. For brevity and clarity, we will use the term calsyntenin-1 throughout the examples.

### EXAMPLE 2

Purification and microsequencing of the 115 kD fragment released from the transmembrane-anchored calsyntenin-1 protein.

For the purification of the 115 kD fragment of calsyntenin-1 that is released from the neurites of spinal cord neurons in the compartmental culture system see example 1), we used the conditioned medium of dissociated cultures of the ventral halves of spinal cords from E6 chickens. 6×106 cells from the ventral halves of the spinal cords of E6 chickens were cultivated in 60 mm collagen-coated culture dishes (porcine collagen, 25010 COL1, Corning, N.Y.) and grown for 7 days with one change of medium. To harvest released proteins, the cells were washed twice with prewarmed MEM without supplements and grown for 2-3 days in serum-free medium with nutrient mixture N3 (Bottenstein and Sato, Proc. Natl. Acad. Sci. USA 1979, 76(1): 514-7), lacking BSA and transferrin. The conditioned medium was harvested, filtrated trough a 0.22 μm filter and stored at −20° C.

For the purification of calsyntenin-1, the conditioned medium was dialyzed against buffer A (20 mM Tris-Cl, pH 8.0), degased, filtrated again through a 0.22 μm filter, and then loaded onto a 1 ml Mono Q anion exchange column (Pharmacia) at a flow rate of 1 ml/min. After washing the column with 10 volumes of buffer A, the proteins were eluted in a gradient from 0% to 50% of buffer B (1 M NaCl in 20 mM Tris-Cl, pH 8.0) within 20 ml. Fractions of 3 ml were collected and analyzed with 2-dimensional SDS-PAGE (O'Farrell, J Biol Chem. May 25, 1975;250(10): 4007-21.) followed by silver staining (Heukeshoven and Dernick, Electrophoresis. 1988;9(1) :28-32.). Calsyntenin-1 was eluted between 300 and 450 mM NaCl.

For preparative separation by 2-dimensional SDS-PAGE, the fractions were pooled and concentrated either according to Wessel and Fluegge, (Anal. Biochem. (1984), 138:141-3)or by centrifugation through a porous membrane (Ultrafree-20, Milipore, Bedford, Mass.). Two-dimensional SDS-PAGE was carried out according to O'Farrell (1975), loading 3-4 concentrated fractions from the anion exchange column onto one gel. The ampholine solution for the isolelectric focusing step was composed of 1.6% pharmalyte 5/8, 0.4% pharmalyte 3/10, and 0.8% pharmalyte 4/6 (all from Pharmacia). The pH range of the gels during isoelectric focusing was from pH 4.9 to 6.8. The second dimension was run on a 7.5% SDS-PAGE gel (Lämmli, 1970).

After Coomassie blue staining, the protein spots with the gel coordinates of calsyntenin-1 were excised and processed by SDS-PAGE using the funnel-well concentration system (Lombard-Platet and Jalinot, Nucleic Acids Res. 1993, 21(17):3935-42). The funnel-well gel electrophoresis system devised by Lombard-Platet and Jalinot (1993) is a method for the concentration of protein from several gel pieces. Two spacers forming a funnel were adapted to the minigel system of Bio-Rad (Bio-Rad, Richmond, Calif.). Sealing was done with 20% acrylamide. The running gel composed of 10% acrylamide had a length of 1 cm, the stacking gel composed of 4% acrylamide was 4-5 cm long.

Prior to loading into the funnel-well, the gel pieces containing calsyntenin-1 were destained from Coomassie blue in 20% ethanol/5% acetic acid and equilibrated for 3 h ar room temperature in sample buffer (4% glycerol, 2.5% SDS, 2.5% β-mercaptoethanol in 25 mM Tris-Cl, pH 6.8). Up to 8 gel pieces containing calsyntenin-1 were carefully transferred into the funnel-well, overlayed with running buffer, and concentrated in the gel for 2 h at 50V. During the concentration, the current changed from 10 mA to 4 mA. The progress of the concentration could be followed by visual inspection of the protein front within the gel due to a schlieren effect. When the protein was concentrated in the middle of the running gel, the run was stopped for in-gel digestion or for transfer onto a PVDF membrane for direct amino acid sequencing. In a typical experiment, a protein band containing approximately 20 μg of calsyntenin-1 was obtained. After excision and destaining with 40% n-propanol (LichroSolv grade, Merck), calsyntenin-1 was extracted with 0.2 M NH₄HCO₃, 50% acetonitrile, and dried in a Speed Vac. For sequencing the N-terminus, concentrated calsyntenin-1 was electrotransferred from the gel onto a PVDF membrane (Immobilon P^{SQ}, Millipore). The calsyntenin-1-containing area on the PVDF membrane was localized by autoradiography and excised. The sequence was determined by Edman degradation on a protein sequencer (Model 477 A, Applied Biosystems, Inc.).

For sequencing of internal peptides, tryptic digestion was carried out within the gel pieces obtained by the funnel-well system (Jeno et al., Anal Biochem. 1995;224(1):75-82.). Calsyntenin-1 was processed as described above by 2-dimensional SDS-PAGE, stained with, Coomassie blue, excised, and concentrated in the funnel-well system. The protein band of approximately 20 μg calsyntenin-1 was cut into small pieces. The gel pieces were destained with 40% n-propanol (LichroSolv grade, Merck), extracted with 0.2 M NH₄HCO₃, 50% acetonitrile, and dried completely in a Speed Vac for 30 min. Tryptic digestion was carried out in digestion buffer (5% acetonitrile in 100 mM Tris-Cl, pH 8.0), containing 1 μg trypsin (Promega, Madison, Wis.; 0.5 μg/μl in 1 mM HCl) and incubated at 37° C. for 18 h. The peptides were extracted with digestion buffer and with 80% acetonitrile, 0.1% trifluoroacetic acid. The pooled extracts were evaporated to obtain the injection volume of the reversed-phase HPLC column (30-50 μl). The peptides were separated in a reversed-phase HPLC column (Vydac C8, 5 μm particle sized, 1 mm (i.d.)×250 mm; Vydac, Hesperia, Calif.) connected to a mass spectrometer (API-III, PE Sciex, Thornhill, Ontario). Solvent A was 0.1% trifluoroacetic acid, solvent B was 80% acetonitrile containing 0.09% trifluoroacetic acid. The elution program used was: 5% solvent B for 5 min; 5% to 60% solvent B during 60 min at a flow rate of 50 μl/min. The effluent was monitored at 215 nm. 90% of the eluted volume were collected and 10% injected online into the mass spectrometer (API-III, PE Sciex, Thornhill, Ontario. The chromatograms were analyzed and single peptide fractions chosen for sequencing. Sequence analysis was performed on a Model G 1005 protein sequencer (Hewlett-Packard, Camas, Wash.), according to the manufacturers protocols.

By this method, the amino acid sequences of the N-terminus and seven internal peptides of the 115 kD fragment of calsyntenin-1 released from the cultures of spinal cord neurons were determined. The following sequences were found:

N-terminal sequence (single letter code for amino acids):

ARVNKHKPWIETTY (Seq. Id. No. 7)

Internal peptides:

Peptide Number Amino acid sequence

Internal peptides:

| Peptide Number | Amino acid sequence |
|---|---|
| 1. | HKPWIETTYHGIVTENDNTVLLDP |
| | (Seq. Id. No. 8) |
| 2. | VEAVDA (Seq. Id. No. 9) |
| 3. | IEYEPGTGSLALFPSMR (Seq. Id. No. 10) |
| 4. | IPDGVVT (Seq. Id. No. 11) |
| 5. | TYKPAEFHW (Seq. Id. No. 12) |
| 6. | EGLDLQIADGV (Seq. Id. No. 13) |
| 7. | GIEMSSSNLGMIITGVDTMASYEEVLHL (Seq. Id. |
| | No. 14) |

The sequence of one internal peptide (Peptide Number 1) overlapped with the N-terminal sequence and, thus, generated an extension of the N-terminal sequence to a length of 29 amino acids, with the following sequence:

ARVNKHKPWIETTYHGIVTENDNTVLLDP

### EXAMPLE 3

Cloning and Sequencing of the Calsyntenin-1 cDNA of the Chicken

The amino acid sequences of the N-terminus and the internal peptides were used to design degenerated primers for RT-PCR using total RNA from B14 chicken brain as template. Total RNA was prepared from E14 chicken brain and from P10 mouse cerebellum (Chomczynski and Sacchi, Anal Biochem. 1987;162(1):156-9.). Oligo(dT)- and random-primed cDNA was produced using M-MLV reverse transcriptase (Promega). For PCR, degenerated primers corresponding to the amino terminus and four internal peptides were synthesized. (sense primers: 5′-GTIAAMAAGCAYAAGCCITGGAT-3′ (Seq. Id. No. 15) and 5′-CATGGIATHGTIACIGAGAATGATAA-3′ (Seq. Id. No. 16); antisense primers: 5′-CCIGTICCIGGCTCATACTCDAT-3′(Seq. Id. No. 17) , 5′-GTATCIACICCITADATDATCATICC-3′ (Seq. Id. No. 18) , 5′-ACICCATCIGCDATCTGIAAATC-3′ (Seq. Id. No. 19)and 5′-GCATCAAACTCIGCCTCCTTATAAAA-3′ (Seq. Id. No. 20). PCR was performed using Taq DNA polymerase (Promega). PCR fragments were sequenced and used for screening cDNA libraries. Approximately 2.5×10⁶ plaques of an oligo(dT)-primed E14 chicken brain cDNA library (Zuellig et al., 1992; Eur. J. Biochem 204(2):453-63), an oligo(dT)-primed P20 mouse brain cDNA library (Stratagene), an oligo(dT)- and random-primed E15 mouse brain cDNA library (Clontech), and an oligo(dT)- and random-primed fetal human brain cDNA library (Clontech), respectively, were screened by hybridization with the corresponding radiolabeled PCR fragments under high stringency conditions (Sambrook et al., 1989; Molecular Cloning. A Laboratory Manual). Positive clones were further characterized.

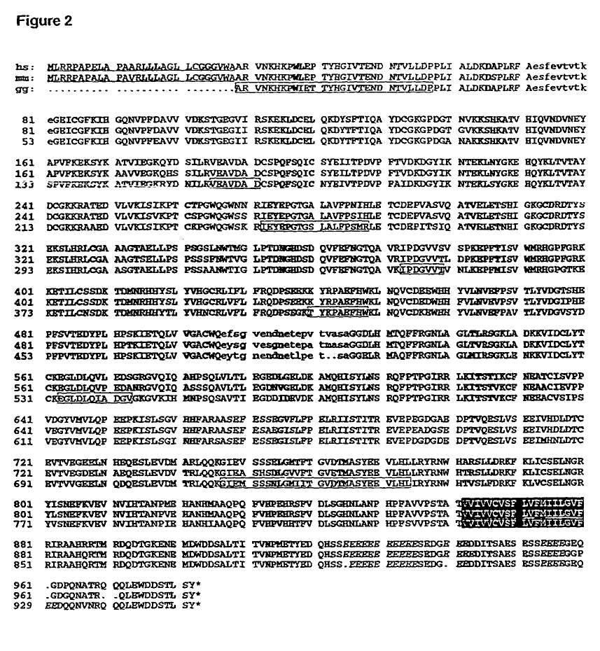

The longest PCR product had a length of 2.2 kb. It contained a single open reading frame (ORF) that encoded all previously determined amino acid sequences (FIG. 2). Screening an oligo(dT)-primed E14 chicken brain cDNA-library (Zuellig et al., 1992; Eur. J. Biochem 204(2):453-63) with this fragment as a probe yielded clones containing additional 3′ sequence of the ORF and the 3′ untranslated region. The composite cDNA contained an ORF of 2850 nt (starting from the amino-terminus of the purified protein). The hydropathy plot provided evidence for a single transmembrane segment of 19 amino acids close to the C-terminus (FIG. 2). Therefore, we concluded that the mature protein was composed of an extracellular N-terminal moiety of 831 amino acids, a transmembrane segment of 19 amino acids, and a cytoplasmic moiety of 100 amino acids. Based on the presumed structural characteristics as a type I transmembrane protein, the 115 kD protein isolated from the supernatant of E6 spinal cord cultures represents the proteolytically cleaved N-terminal fragment of the full-length transmembrane protein. The exact location of the cleavage site within the sequence of full-length calsyntenin-1 remains to be determined. Based on the location of the tryptic peptides (boxed in gray in FIG. 2), the released fragment isolated from the culture supernatant must have a length of at least 750 amino acids (as counted from the N-terminus of the mature protein).

Thirty-eight of the 100 amino acids of the cytoplasmic segment of calsyntenin-1 are acidic (see Table 1). In the most acidic middle part, 18 out of 20 residues are acidic and the flanking sequences are enriched in acidic residues as well. Similarly acidic segments are characteristic for calsequestrin, calreticulin, and protein disulfide isomerase (Fliegel et al., J. Biol. Chem. 1989; 264(36):21522-28). These proteins are essential for the storage of Ca²⁺ in the sarcoplasmic reticulum of skeletal muscle cells and the endoplasmic reticulum of nonmuscle cells, due to their capacity to bind large numbers of Ca²⁺ ions with low affinity (Baksh and Michalak, J Biol Chem. 1991; 266(32):21458-65; Ohnishi and Reithmeier, iochemistry. 1987; 26(23):7458-65.).

### EXAMPLE 4

Cloning and Sequencing of the Calsyntenin-1 cDNA of the Human and the Mouse: Species Homologues of Calsyntenin-1 in Vertebrates Exhibit a High Degree of Structural Conservation

The major part of the cDNA of human calsyntenin-1 was found by searching the THC (Tentative human consensus sequence) database with the THC Blast program. Seven THCs (THC176438, THC178825, THC195843, THC200424, THC192325, THC211114, and THC211115) with homology to the cDNA of chicken calsyntenin-1 were identified and used to compose a partial sequence of the human cDNA lacking a segment of the 5′ end and two internal segments. The gaps were closed by RT-PCR. The putative translation start codon and a segment of 5′ UTR sequence were found by screening a human brain cDNA library. Thus, we obtained a human cDNA sequence with an ORF of 2943 bp that was 100% identical with KIAA0911, a cDNA resulting from a screen for brain-specific proteins that was not further characterized (Nagase et al., DNA Res. 1998; 5 (6), 355-364).

The cDNA of mouse calsyntenin-1 was obtained by RT-PCR and subsequent screening of brain cDNA libraries. Based on the sequence of overlapping clones, a single ORF of 2937 nt, encoding a peptide of 979 amino acids was defined (FIG. 2).

The sequences of human and mouse calsyntenin-1 starting with the amino acid 29 correspond to the sequence of the N-terminal peptide of chicken calsyntenin-1 (FIG. 2). The deduced amino acid sequences of the human and the mouse orthologs had an identity of 86.4% and 84.7%, respectively to chicken calsyntenin-1 (Table I). The amino acid sequence identity of human and mouse calsyntenin-1 was 92%.

These results revealed that the species homologues of calsyntenin-1 in three vertebrate species, namely human, mouse, and chicken, exhibit a high degree of structural conservation. Based on the high structural conservation, a high degree of functional conservation among the species orthologs of calsyntenin-1 of different vertebrate species, such as human, mouse, rat, and chicken, can be expected.

### EXAMPLE 5

Database Searches for Calsyntenin-1-Related Genes: Related Sequences Found in Databases Suggest the Existence of Calsyntenin-Like Genes in D. melanogaster and C. elegans

Genes with a structural relationship to vertebrate calsyntenin-1 were also found in the databases for D. melanogaster and C. elegans. In the Genome Annotation Database of Drosophila (GadFly), a single calsyntenin-1 -like gene was found (acc. Nr. GC11059). Based on six overlapping ESTs (GM09293, HL03914, LD07408, LD 11689, GM10465, LD06216) we have determined the sequence of the corresponding cDNA (acc. Nr. AJ289018). The deduced protein exhibits an amino acid sequence identity of approximately 35% with vertebrate calsyntenin-1. A further calsyntenin-1-related gene, B0034.3 with Accession. Nr. AAC38816, was found in C.elegans (see Table I).

These results indicate that calsyntenin-1-like genes are also found in invertebrates. Thus, calsyntenin-1 represents an evolutionarily ancient gene that has been well conserved throughout evolution.

### EXAMPLE 6

Calcium-Binding Studies with the Cytoplasmic Segment of Calsyntenin-1: The Cytoplasmic Segment of Calsyntenin-1 Binds Calcium Ions

The clustered occurrence of acidic amino acids is a typical trait of high-capacity, low-affinity Ca²⁺-binding proteins found in vesicular Ca²⁺ stores, such as calsequestrin (Yano and Zarain-Herzberg, Mol Cell Biochem. 1994; 135(1):61-70.) and calreticulin (Krause and Michalak, Cell. 1997; 88(4):439-43). To test for the Ca²⁺-binding capacity of calsyntenin-1, we generated a fusion protein of its cytoplasmic segment with the bacterial protein intein. A fusion protein of the cytoplasmic segment of mouse calsyntenin-1 and an N-terminal intein tag was expressed in bacteria using the Impact-CN system (New England Biolabs Inc.).

The cDNA of the cytoplasmic segment of calsyntenin-1 was amplified by PCR before it was inserted in frame in the multiple cloning region (MCS) of the pTYB11 vector (New England BioLabs, Inc.). The PCR was performed using the proofreading polymerase Pwo (Roche), the complete mouse cDNA of calsyntenin-1 as template and the primers LV38Fmax3 (5′-GGGGAACAGAAGAGCTGCACATCAGCGAACG-3′) (Seq. Id. No. 21) and LV39Bmax3 (5′-CCCCCTCGAGTTAGTAGCTGAGTGTGGAG-3′) (Seq. Id. No. 22). The PCR fragment was cloned into the MCS of pTYB11 using restriction sites SapI and XhoI. After ligation the plasmid was used to transform competent E. coli strain BL21DE. A single colony containing the correct plasmid was used for protein expression. One liter LB medium containing 100 μg/ml ampicillin was inoculated with a fresh colony. The culture was incubated in an air shaker at 37° C. until the OD₂₆₀ reached 0.6. Afterwards the culture was transferred to a 22° C. shaker. The protein expression was induced by adding 0.5 mM Isopropyl-B-D-Thiogalactoside (IPTG). After 6 h, the cells were spun down at 5000×g for 10 minutes at 4° C. The cell pellet was resuspended in 20 ml of cell lysis buffer (20 mM Tris; 500 mM NaCl; pH 8.0) and the cells were broken by sonication. To obtain a clarified cell extract the crude cell extract was centrifuged at 12000×g for 30 minutes. To purify the calsyntenin-1/intein fusion protein the clarified cell extract was loaded onto a chitin column and washed with a high flow rate (2 ml/min) and stringent wash conditions (1 M NaCl). The fusion protein was eluted with 3×SDS-PAGE sample buffer (187.5 mM Tris-HCl pH6.8; 6% SDS; 30% glycerol and 0.03% bromphenolblue) and incubation for 3 minutes at 99° C.

As a control maltose-binding protein (pMYB5 control plasmid; New England BioLabs, Inc.) fused to the intein tag was used.

The Ca²⁺-binding assay was performed as described previously (Maruyama et al., J Biochem 1984; 95(2):511-9.). For SDS-PAGE, 5 μg of purified fusion protein were heated at 100° C. for 5 min prior to loading on 10% polyacrylamide gels. The electrotransfer onto a nitrocellulose membrane (Schleicher & Schuell, Dassel, Germany) was performed at a constant voltage of 100 V for 1 h at 4° C., using a solution containing 20% methanol, 0.025 M Tris-Cl, and 0.129 M glycine (pH8.5) as the electrode buffer. After transfer, the membrane was soaked in a solution containing 60 mM KCl, 5 mM MgCl₂, and 10 mM imidazole-HCl, pH6.8, and the buffer was exchanged several times in an hour. Then, the membrane was incubated in the same buffer containing 0.5 μM, or 1 μM, or 5 μM, respectively, of ⁴⁵Ca²⁺ (28 mCi/mg calcium , Amersham, Buckinghamshire, UK) for 10 min. The membrane was rinsed with distilled water for 2 minutes. Excess water was absorbed with Whatman No. 1 filter paper and the membrane was dried at room temperature. For autoradiography, the blots were analyzed using a PhosphorImager (Molecular Dynamics, Sunnyvale, Calif.).

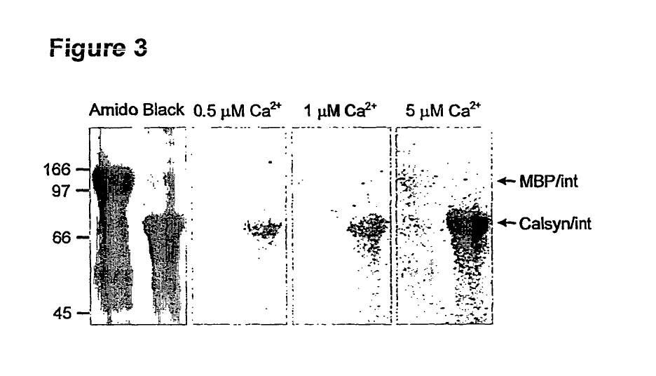

As shown in FIG. 3, a dose-dependent ⁴⁵Ca²⁺ signal overlapping with the calsyntenin-1/intein fusion protein was found when the nitrocellulose filters were incubated with Ca²⁺ concentrations of 0.5 μM, 1 μM, and 5 μM. No Ca²⁺ binding was observed with a fusion protein composed of the bacterial maltose-binding protein and intein (MBP/intein in FIG. 3).

With the calsyntenin-1/intein fusion protein, we observed an extensive precipitation when CaCl₂ was added. The addition of 50 mM or more Ca²⁺ caused the soluble calsyntenin-1/intein, but not MBP/intein, to precipitate (not shown). The Ca²⁺-induced precipitation of calsyntenin-1/intein was prevented by the addition of an equimolar concentration of EDTA. Because precipitation was not observed at Ca²⁺ concentrations below 50 mM, we concluded that cross-bridging between cytoplasmic domains of calsyntenin-1 involves low-affinity binding of Ca²⁺ to sites which are clearly distinct from the high-affinity sites detected by ⁴⁵Ca²⁺ binding on the nitrocellulose membranes. With respect to the low-affinity binding capacity for Ca²⁺, the cytoplasmic domain of calsyntenin-1 exhibits striking similarities with calsequestrin, the major calcium-binding protein of the sarcoplasmic reticulum of striated muscle cells. Calsequestrin exhibits a similar clustering of acidic residues cumulating in a contiguous stretch of 14 acidic residues. The Ca²⁺-binding capacity of peptides with contiguous acidic residues has been linked to a general cation-binding capacity rather than specific Ca²⁺ sites. A comparison of proteins with such acidic stretches suggested that the Ca²⁺-binding capacity was proportional to the content of acidic residues (Lucero et al., J Biol Chem. 1994; 269(37):23112-9). X-ray crystallography suggested that the low-affinity binding of Ca²⁺ occured via intercalation of Ca²⁺ between the acidic C-terminal segments of calsequestrin dimers (Wang et al., Nat Struct Biol. 1998; 5(6):476-83). Similarly, calsyntenin-1 may bind Ca²⁺ by intercalation between its cytoplasmic moieties which are held in an ordered parallel orientation by transmembrane anchorage.

In summary, these results indicate that the cytoplasmic domain of calsyntenin-1 exhibits both high-affinity and low-affinity binding of Ca²⁺.

### EXAMPLE 7

Northern Blot Analyses of the Tissue Distribution of Calsyntenin-1 mRNA: The Brain is the Tissue with the Highest Expression Level of Calsyntenin-1 mRNA.

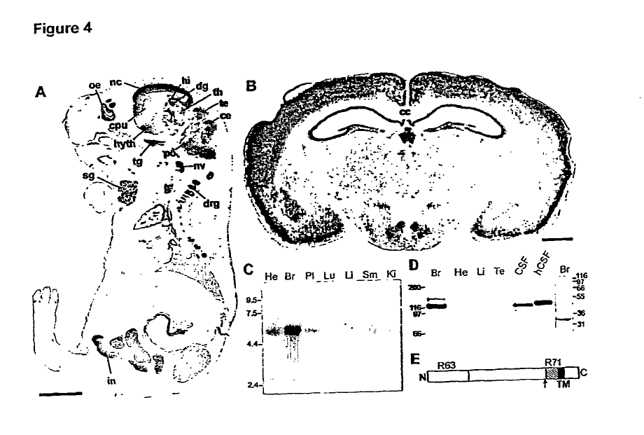

In order to obtain information on the expression pattern of calsyntenin-1 in different human tissues, a human multiple-tissue Northern blot (Clontech) was hybridized with a 2.8 kb cDNA fragment of human calsyntenin-1 labeled with [α-³²P] dCTP (Amersham) using the Prime-it II random primer labeling kit (Stratagene). Hybridization was performed at 42° C. overnight and the hybridization signals were analyzed with a PhosphorImager (Molecular Dynamics). Northern blot analysis of poly(A)-enriched RNA from adult human tissues revealed a single species of calsyntenin-1 mRNA of approximately 5 kb (FIG. 4C). The highest expression of calsyntenin-1 mRNA was observed in brain. Low signals were detected in heart, placenta, skeletal muscle, and kidney. No transcript was found in lung and liver.

Therefore, these results clearly demonstrate that the highest expression levels of calsyntenin-1 mRNA is found in the brain.

### EXAMPLE 8

In Situ Hybridization Analyses of the Tissue Distribution of Calsyntenin-1 mRNA: Calsyntenin-1 is Predominantly Expressed in Neurons

In order to determine the expression of calsyntenin-1 in the brain at cellular resolution, in situ hybridization was performed as described previously (Schaeren-Wiemers and Gerfin-Moser, Histochemistry 1993; 100(6): 431-40). In situ hybridization on cryosections from a E18 mouse revealed a strong cellular expression of calsyntenin-1 mRNA in the gray matter of the central and the peripheral nervous system (FIG. 4A). In a saggital section of an E18 mouse the following regions were labeled (FIG. 4A), the neocortex (nc), the hippocambal formation (hi), the caudate putamen (cpu), the thalamus (th), the hypothalamus (hyth), the cerebellum (ce), the pons (po), the trigeminal ganglion (tg), the dorsal root ganglia (drg), the olfactory epithelium (oe), the submandibular gland (sg) and the intestine (in). No calsyntenin-1 mRNA was detected in non-neural tissues, except in the submandibular gland. Control sections processed with the sense probe, showed no staining ( ). In the adult mouse, calsyntenin-1 mRNA was abundant in all areas of the gray matter (FIG. 4B). In the white matter, such as the corpus callosum (cc) no calsyntenin-1 expression was found. Inspection at higher magnification indicated a neuronal expression pattern in all areas of the CNS and the PNS. Most, if not all neurons, expressed calsyntenin-1 mRNA, yet considerable differences in the expression level were found. Northern blot analysis of calsyntenin-1 mRNA in adult human tissues is shown in FIG. 4C. Two μg of purified polyA⁺ RNA per lane form heart (He), brain (Br), placenta (Pl), lung (Lu), liver (Li), skeletal muscle (Sm), and kidney (Ki) were analysed with radiolabeled cDNA fragments of human calsyntenin-1. The molecular size scale is in kb. In FIG. 4D is shown a Western blot analysis of chicken calsyntenin-1 protein. 150 μg of tissue extract from adult chicken brain (Br), heart (He), liver (Li), testis (Te), chicken cerebrospinal fluid (CSF), and human cerebrospinal fluid (hCSF) were subjected to SDS-PAGE and immunoblotting using polyclonal antibodies R63 (left panel) and R71 (right panel) against calsyntenin-1. The molecular weight scale is in kD. FIG. 4E shows a schematic drawing indicating the proteolytic cleavage site (arrow) on the calsyntenin-1 protein and the location of the recombinant peptide segments used for raising the R63 (shadowed) and the R71 (hatched) antibodies in the complete sequence of mature calsyntenin-1. Note that antibody R63 recognizes both the full-length form of calsyntenin-1 and the N-terminal cleavage product. In contrast, antibody R71 recognizes the transmembrane stump generated by the proteolytic cleavage of calsyntenin-1. The transmembrane domain (TM) is marked in black. Scale bars: (A), 2.5 mm; (B), 1.0 mm.

### EXAMPLE 9

Characterization of Calsyntenin-1 Protein and Its Cleavage Products: Calsyntenin-1 Protein Occurs as a Full-Length Transmembrane Protein, a Membrane-Bound C-Terminal Cleavage Product, and a Soluble N-Terminal Cleavage Product

To analyze the tissue distribution of full-length calsyntenin-1 and its cleavage products two antibodies, termed antibody R63 and R71, respectively, were raised in rabbits. The immunogen for the R63 antiserum consisted of a 267 amino acid peptide starting at the N-terminus of chicken calsyntenin-1. The immunogen for the R71 antiserum consisted of an 87 amino acid peptide located immediately outside of the transmembrane segment of chicken calsyntenin-1. Both fragments were expressed with a His-tag in bacteria and purified using a NiNTA column (Qiagen).

Production of the R63 Antigen:

The cDNA fragment of the 267 amino acids long peptide located at the N-terminus of chicken calsyntenin-1 was amplified by PCR before it was inserted in frame in the pTFT74 vector. The PCR was performed using the proof-reading polymerase pfu (Stratagene), the cDNA of chicken calsyntenin-1as template and the primers LV31Fchax3 (5′-GGGCCATGGCTCGTGTTAACAAGCATAAGCCCTGGATTG-3′) (Seq. Id. No. 23) and LV32Bchax3 (5′-CCCAAGCTTAGTGGTGGTGGTGATGGTGTGGTTCATCACATGTGTCC-3′) (Seq. Id. No. 24). The PCR fragment was cloned into the pTFT74 vector using restriction sites NcoI and HindIII. After ligation the plasmid was transformed into competent E. coli strain BL21DE. A single colony containing the correct plasmide was used for protein expression. 1 liter LB medium containing 100 μg/ml ampicillin was inoculated with a fresh colony. The culture was incubated in an air shaker at 37° C. until the OD₂₆₀ reached 0.6. Afterwards the culture was transferred to a 22° C. shaker. The protein expression was induced by adding 0.5 mM IPTG. After 6 h the cells were spun down at 5000×g for 10 minutes at 4° C. The cell pellet was resuspended in 20 ml of cell lysis buffer (50 mM Tris; pH 8.0) and the cells were broken by sonication. To obtain a clarified cell extract the crude cell extract was centrifuged at 12000×g for 30 minutes. The fusion protein was purified using a NiNTA column (Qiagen) according to the instruction manual. The R63 antigen generated in this way is shown below (single letter code for amino acids; capital letters indicate amino acids found in calsyntenin-1; small letters indicate the initial methionine and the histidine-tag, respectively.

Protein sequence of R63 antigen (Seq. Id. No.

ARVNKHKPW IETTYHGIVT ENDNTVLLDP
PLIALDKDAP LRFAESFEVT VTKEGEICGF KIHGQNVPFE
AVVVDKSTGE GIIRSKEKLD CELQKDYTFT IQAYDCGKGP
DGANAKKSHK ATVHIQVNDV NEYSPVFKEK SYKATVIEGK
RYDNILKVEA VDADCSPQFS QICNYEIVTP DVPFAIDKDG
YIKNTEKLSY GKEHQYKLTV TAYDCGKKRA AEDVLVKISI
KPTCKPGWQG WSKRIEYEPG TGSLALFPSN RLETCDEP

Production of the R71 Antigen:

The cDNA fragment of the 87 amino acid long peptide used as antigen for generation of R71 antibody was amplified by PCR before it was inserted in frame in the pTFT74 vector. The PCR was performed using the proof-reading polymerase pfu (Stratagen), the cDNA of chicken calsyntenin-1as template and the primers MS1Fchax3 (5′-GGGCCATGATACGCTACAGAAACTGGCAC-3′)(Seq. Id. No. 26) and MS2Bchax3 (5′-CCCAAGCTTAGTGGTGGTGGTGATGGTGAGTGGCTGTACTTGGAACAAC-3′)(Seq. Id. No. 27). The PCR fragment was cloned into the pTFT74 vector using restriction sites NcoI and HindIII. After ligation the plasmid was transformed into-competent E. coli strain BL21DE. A single colony containing the correct plasmide was used for protein expression. 1 liter LB medium containing 100 μg/ml ampicillin was inoculated with a fresh colony. The culture was incubated in an air shaker at 37° C. until the OD₂₆₀ reached 0.6. Afterwards the culture was transferred to a 22° C. shaker. The protein expression was induced by adding 0.5 mM IPTG. After 6 h the cells were spun down at 5000×g for 10 minutes at 4° C. The cell pellet was resuspended in 20 ml of cell lysis buffer (50 mM Tris; pH 8.0) and the cells were broken by sonication. To obtain a clarified cell extract the crude cell extract was centrifuged at 12000×g for 30 minutes. The fusion protein was purified using a NiNTA column (Qiagen) according to instruction manual. The R71 antigen generated in this way is shown below (single letter code for amino acids; capital letters indicate amino acids found in calsyntenin-1; small letters indicate the initial methionine and the histidine-tag, respectively.

Protein sequence of R71 antigen (Seq. Id. No.

mIRYRNWHTV SLFDRKFKLV CSELNGRYVS
NEFKVEVNVI HTANPIEHAN HIAAQPQFVH PVHHTFVDLS
GHNLANPHPF SVVPSTATgh hbhhh

The antisera against the proteins (antigen R63 and antigen R71) were raised in rabbits by injection of 50 μg protein in phosphate-buffered saline with complete Freund's adjuvans for the first injection and with incomplete Freund's adjuvans for the booster injections. The anti-calsyntenin-1 antibodies were affinity purified from the immuneserum by a passage over a protein-G column followed by an antigen-conjugated column.

Antibody R63, raised against the N-terminal 267 amino acids of the mature proteins detects both full-length calsyntenin-1 as well as the N-terminal part resulting from the proteolytic cleavage (FIG. 4E). Antibody R71, raised against a segment of 87 amino acids located adjacent to the transmembrane domain, detects the trans-membrane stump generated by the proteolytic cleavage. In Western blots, calsyntenin-1 immunoreactive bands were found exclusively in brain extracts and in the cerebrospinal fluid (CSF; FIG. 4D). Extracts of all the other tissues that were tested, including heart, liver, testes (FIG. 4D), as well as kidney, lung, and spleen (not shown) did not exhibit calsyntenin-1 immunoreactivity. In brain extracts of adult chickens, two bands with apparent MWs of 150 and 115 kD were found with antibody R63, whereas a single band at 33 kD was detected with antibody R71 (FIG. 4D). The 115 kD band comigrated with the protein initially identified with the compartmental culture system as a released protein of the axo-dendritic compartment of spinal cord neurons. The 150 kD band represents most likely the full-length form of calsyntenin-1, based on the estimated size of the released fragment and the length of the transmembrane and cytoplasmic segments. In the cerebrospinal fluid, antibody R63 recognized only a single band of 115 kD, that corresponds to the soluble N-terminal cleavage product of calsyntenin-1. In contrast to brain extract, CSF did not contain full-length calsyntenin-1 or the transmembrane stump. Taken together, these results indicate that full-length calsyntenin-1 and its cleavage products coexist in brain tissue. The N-terminal 115 kD fragment of calsyntenin-1 that is solubilized after proteolytic cleavage is also found in the CSF.

An investigation of human CSF indicated that human calsyntenin-1 is cleaved in the extracellular moiety the same way as calsyntenin-1 of other vertebrates. As demonstrated in FIG. 4E, a prominent calsyntenin-1-immunoreactive band with the same molecular weight as the N-terminal cleavage fragment of calsyntenin-1 found in mouse and chicken. These results indicate that the proteolytic cleavage of calsyntenin-1 in the extracellular domain is a characteristic that was conserved during evolution.

### EXAMPLE 10

Studies of the Subcellular Localization of Calsyntenin-1 Protein at the Light Microscopic Level: Cell Surface-Bound Calsyntenin-1 is Colocalized with Established Synaptic Marker Proteins

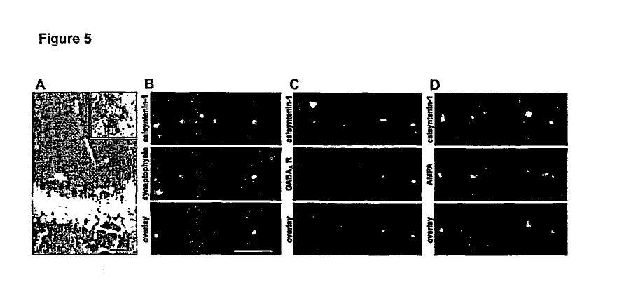

Immunoperoxidase staining of tissue sections of the hippocampus (FIG. 5A) and the cerebral cortex (not shown) revealed that calsyntenin-1 was abundant in synapse-rich regions. At higher magnification, a punctate appearance of the immunostaining in the neuropil (insert of FIG. 5A) was found, suggesting a synaptic localization of calsyntenin-1.

A detailed study of the subcellular location of cell surface-associated calsyntenin-1 was performed by immunofluorescence colocalization in cultures of dissociated hippocampal neurons. Cell suspensions of hippocampi dissected from brains of E17 mice were prepared by digestion with trypsin (0.25% for 10 min at 37° C.) and trituration using a blue Gilson tip. Cells were then plated onto acid-washed, poly-L-lysine-treated glass coverslips or poly-L-lysine-treated plastic dishes in DMEM supplemented with B27 (Gibco/Life Technologies), 0.25 mg/ml Albumax (Gibco/Life Technologies), 2 mM glutamine, and 0.1 M sodium pyruvate. Cultures were maintained for up to 4 weeks in a humidified incubator with 5% CO₂ at 37° C.

Cells were fixed in 4% paraformaldehyde and 4% sucrose in PBS for 30 min at 37° C. After rinsing with PBS, cells were preincubated in 10% fetal calf serum and 0.1% glycine in PBS at room temperature for 1 h before incubation with the primary antibody in 3% fetal calf serum in PBS at 4° C. for 24-48 h. For the double-labeling experiments, primary antibodies were incubated together. Cells were washed for at least 30 min in three changes of PBS. For secondary antibodies FITC-conjugated goat anti-rabbit IgG (Cappel) and Cy3-conjugated donkey anti-mouse IgG (Jackson Immuno Research Laboratories, Inc.) were used. For stainings with anti-GluR2, the cells were permeabilized with 0.1% saponin.

Established synaptic markers, such as synaptophysin, the α₂ subunit of the GABA_{A} receptor, and the GluR2 subunit of the AMPA receptor were used as markers for presynaptic terminals and postsynaptic membranes, respectively. The antibody against the GABA_{A} receptor subunit α2 was provided by Jean-Marc Fritschy. The antibodies against synaptophysin, PSD95, GluR1 and GluR2 were from Roche, Pharmingen, and Chemicon, respectively. As demonstrated in FIG. 5, B-D, calsyntenin-1 immunoreactivity exhibited a patchy pattern along neurite bundles. A very similar staining pattern was found with the antibodies against synaptophysin (FIG. 5B) and the GABA_{A} receptor (FIG. 5C). In the overlay, the majority of the large areas labeled with antibodies against synaptophysin and the GABA_{A} receptor were at least partially superposed, with the calsyntenin-1 immunoreactivity. With a commercially available antibody againt a cytoplasmic epitope of GluR2, which required permeablization of the cells and, therefore, stained both surface-exposed and internal AMPA receptors (FIG. 5D), large immunoreactive patches were found in close proximity to and sometimes partially overlapping with patches of calsyntenin-1 immunoreactivity. Together, these results demonstrate the synaptic localization of calsyntenin-1.

### EXAMPLE 11

Studies of the Subcellular Localization of Calsyntenin-1 Protein by Immuno-Electron Microscopy: Full-Length Calsyntenin-1 is a Component of the Postsynaptic Membrane.

To reveal the subcellular localization of calsyntenin-1 in CNS neurons, we have used preembedding and postembedding immuno-electronmicroscopy. To prepare brain tissue for immuno-EM, 8 adult Wistar and OFA line rats (200-250 g) of both sexes were deeply anaesthetized with metiofane (methoxyflurane, Pitman-Moore Inc., USA) and perfused through the ascending aorta for 15-25 min first with 0.9% saline for 1 min followed by fixative containing 3.5-4% paraformaldehyde, 0.015-0.05% glutaraldehyde, and 0.2% picric acid made up in 0.1M phosphate buffer pH 7.4. Then brains were removed from the skull into cold PB and either 70 μm (6 rats used in preembedding immunocytochemistry) or 500 μm thick coronal sections (2 rats used for freeze substitution) were cut on a vibratome.

For preembedding immunocytochemistry, the sections were cryoprotected in 30% sucrose, quickly frozen in liquid nitrogen and thawed in PB. After preincubation in 20% normal goat serum (NGS; Vector Labs, USA), sections were incubated in primary antibody made up in 0.05 mM Tris buffered saline pH 7.4 (TBS) containing 2% BSA and 2% NGS at 4° C. for 2 days. For immunogold method, sections were incubated overnight in 1:40 goat anti-rabbit IgG coupled to 1.4 nm gold (Nanoprobes Inc. Stony Brook, N.Y.), postfixed in 1% glutaraldehyde in PBS followed by silver enhancement of the gold particles with an HQ Silver kit (Nanoprobes Inc). For peroxidase reaction, sections were incubated for 4 h at RT in biotinylated goat anti-rabbit IgG (Vector Labs) diluted 1:200 in TBS containing 1% NGS followed by 2 h incubation in avidin-biotin-peroxidase complex (ABC kit; Vector Labs) diluted 1:100 in TBS. Antigenic sites were revealed using standard 3,3′-diaminobenzidine tetrahydrochloride histostaining procedure (0.05% DAB and 0.01% H₂O₂ in TB pH 7.6). The gold-silver and peroxidase reacted sections were post-fixed in 1% osmium tetroxide in PB, stained with 2% uranyl acetate, dehydrated in graded series in ethanol and flat-embedded on glass slides in Durcupan ACM resin (Fluka) for electron microscopy.

For postembedding immunocytochemistry on ultrathin section, we used the freeze substitution and low temperature embedding procedure as described earlier (Baude et al., Neuron. 1993; 11(4):771-87). Vibratome sections were cryoprotected in 1 M sucrose, frozen on a Reichert MM80E device, dehydrated in methanol at −80° C. and embedded in Lowicryl HM 20 (Chemische Werke Lowi GmBH, Germany) using Leica CS auto apparatus. Ultrathin sections 80 nm thick from Lowicryl embedded blocks were picked up on nickel grids and incubated for 30 min on drops of blocking solution conisting of 1% BSA, 0.1% cold-water fish skin gelatine (Sigma), and 5% NGS in TBS containing 0.1% Triton X-100. The blocking solution was also used for diluting the primary and secondary antibodies. The grids were incubated overnight in primary antibodies (16-24 μg/ml) followed by 2 h incubation on drops of goat anti-rabbit IgG coupled to 1.4 nm gold (Nanoprobes Inc.) diluted 1:80. The antibodies were fixed with 2% glutaraldehyde for 4 min prior to silver enhancement with an HQ kit (Nanoprobes Inc.) for 3-5 min. Then sections were contrasted for electron microscopy with saturated aqueous uranyl acetate followed by lead citrate. For double-sided immunoreaction, sections were etched with sodium ethanoate for 2-3 s prior to immunoincubation (Matsubara et al., Dev. Biol.1996; 180(2): 499-510).

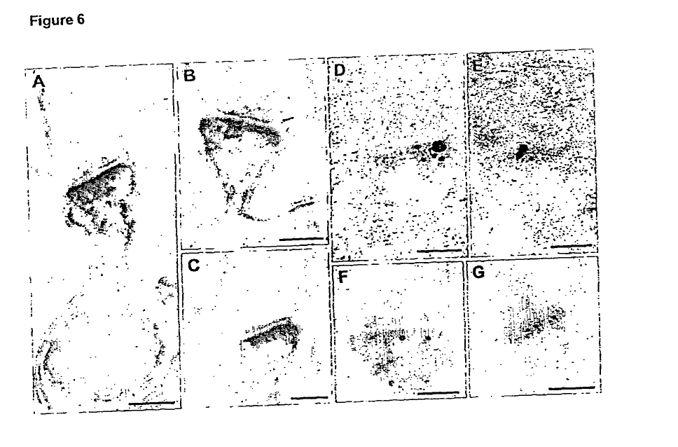

Both preembedding and postembedding immuno-EM demonstrated unequivocally that calsyntenin-1 is located in the postsynaptic membrane of both excitatory and inhibitory synapses. Preembedding immuno-EM with peroxidase-labeled antibodies located calsyntenin-1 in the postsynaptic membrane of synapses located on dendritic spines, dendritic shafts, and on neuronal somas (FIG. 6, A-C). In some synapses, calsyntenin-1 immunoreactivity was also found over part of the adjacent perisynaptic membranes. Rarely, floccular immunoreactivity was found in dendritic spines. Postembedding immunogold staining of rat hippocampus embedded at low temperature confirmed the localization of calsyntenin-1 in the postsynaptic membrane (FIG. 6, D-G). Both asymmetric synapses with round vesicles and thick PSDs (Type 1 according to Gray, 1959) and symmetric synapses with pleomorphic vesicles and thin PSDs (Type 2) exhibited calsyntenin-1 immunoreactivity, confirming calsyntenin-1 as a component of the postsynaptic membrane in both excitatory and inhibitory synapses.

In consideration of the postsynaptic localization of calsyntenin-1 (as shown in the present example), the calcium-binding capacity of the cytoplasmic segment of calsyntenin-1 bcomes particularly interesting. Our studies provide evidence for the presence of both high-affinity and low-affinity Ca²⁺-binding sites. We found Ca²⁺-binding to the cytoplasmic domain of calsyntenin-1 at a concentration as low as 0.5 μM. Therefore, the cytoplasmic domain of calsyntenin-1 binds Ca²⁺ at concentrations occurring during postsynaptic Ca²⁺ influx, suggesting calsyntenin-1 as a modulator of postsynaptic Ca²⁺ signals. In parallel to the high-capacity, low-affinity Ca²⁺-binding function of calsequestrin, which exhibits a similar clustering of acidic residues cumulating in a contiguous stretch of 14 acidic residues, the cytoplasmic domain of calsyntenin-1 may also have the capacity for low-affinity Ca²⁺ binding. The Ca²⁺-binding capacity of peptides with contiguous acidic residues has been linked to a general cation-binding capacity rather than specific Ca²⁺ sites. A comparison of proteins with such acidic stretches suggested that the Ca²⁺-binding capacity was proportional to the content of acidic residues (Lucero et al., J Biol Chem. 1994; 269(37):23112-9). X-ray crystallography suggested that the low-affinity binding of Ca²⁺ occured via intercalation of Ca²⁺ between the acidic C-terminal segments of calsequestrin dimers (Wang et al., Nat Struct Biol. 1998; 5(6):476-83). Similarly, calsyntenin-1 may bind Ca²⁺ by intercalation between its cytoplasmic moieties which are held in an ordered parallel orientation by transmembrane anchorage.

Due to its anchorage in the postsynaptic membrane, the cytoplasmic domain of calsyntenin-1 establishes a fixed Ca²⁺ buffer beneath the postsynaptic membrane. Fixed buffers, in contrast to mobile buffers, restrict the diffusion of Ca²⁺ (Kasai and Petersen, Trends Neuroscience 1994; 17(3): 95-101). They also decrease the peak values of free Ca²⁺ and, by delayed release of Ca²⁺, prolong Ca²⁺ elevations. As a fixed Ca²⁺ buffer, calsyntenin-1 may temporarily retain Ca²⁺ in the subsynaptic zone and retard its dissipation. In this role, calsyntenin-1 may potentially be a modulatory element in synaptic processes where transient increases in intracellular Ca²⁺ are of crucial importance, such as LTP (Bliss and Collingridge, Nature. 1993; 361(6407):31-9), LTD (Linden and Connor, Annu Rev Neurosci. 1995; 18:319-57), as well as in coincidence detection within dendritic spines (Zucker, Curr Opin Neurobiol. 1999; 9(3):305-13). Post-synaptic Ca²⁺-transients have been reported to trigger either LTP or LTD, depending on the concentration and the duration of the Ca²⁺ change. High elevations of Ca²⁺ for a few seconds induce LTP, whereas lower elevations of Ca²⁺, lasting for a longer time span of approximately 1 min, were found to induce LTD (Malenka et al., Neuron. 1992; 9(1):121-8). The presence or absence of a Ca²⁺ buffer in the subsynaptic space could, therefore, be an important element in the mechanism determining whether the outcome of a Ca²⁺ transient is LTP or LTD. A recently reported coincidence detection mechanism in dendritic spines of cortical or hippocampal pyramidal neurons (Koester and Sakmann, Proc Natl Acad Sci USA. 1998; 95(16):9596-601) generates a non-linear summation of Ca²⁺ signals, if an afferent input and a backpropagating action potential (AP) arrive at a synapse within a time window of 200 ms. When the afferent input is followed by a backpropagating dendritic AP, a supralinear summation of Ca²⁺ signals is found. In contrast, a decreased Ca²⁺ influx results when the backpropagating AP preceeds the afferent input. The enhancement of the Ca²⁺ signal that occurs when the AP follows the EPSP has been attributed to a voltage-dependent relief of the Mg²⁺ block of the NMDA receptor, whereas a Ca²⁺-dependent NMDA-receptor inactivation has been proposed as the mechanism underlying the reduced Ca²⁺ influx when the AP arrives first at the synapse (for a review see (Zucker, Curr Opin Neurobiol. 1999; 9(3):305-13)). In both processes, a fixed buffer beneath the post-synaptic membrane could play a role. By its high-affinity Ca²⁺ binding, calsyntenin-1 might contribute to supralinear Ca²⁺ signaling. It has been suggested that buffer saturation may be an important “invisible” component in the mechanisms generating supralinear additivity of Ca²⁺ signals (Neher, Cell Calcium 1998; 24(5-6):345-57). When Ca²⁺ influx through NMDA- and voltage-gated Ca²⁺ channels coincides, more free Ca²⁺ may be generated, because the Ca²⁺ buffers are saturated by the first type of influx. By low-affinity binding of Ca²⁺ beneath the postsynaptic membrane, calsyntenin-1 could prolong Ca²⁺ transients, resulting in enhanced Ca²⁺-dependent NMDA-receptor inactivation and, thus, a prolonged window of sublinear Ca²⁺ signaling. In a recent study with cerebellar Purkinje cells, supralinear Ca²⁺ signaling has been attributed to the saturation of a mobile high-affinity Ca²⁺ buffer (dissociation constant 0.37 μM) and to a contribution of an immobile low-affinity buffer (Maeda et al., Neuron. 1999; 24(4):989-1002). In that study, modulations of Ca²⁺ influx or Ca²⁺ release from internal stores were excluded as the major source for the supralinearity in the Ca²⁺ responses of the Purkinje cells; rather, the supralinear responses were attributed to be predominantly due to saturation of the mobile high-affinity buffer. The immobile low-affinity buffer was suggested to contribute by prolonging the presence of Ca²⁺ and, thus, broaden the time-window of supralinear summation (Maeda et al., Neuron. 1999; 24(4):989-1002). Calsyntenin-1, by combining both high- and low affinity Ca²⁺ buffering in one molecule at a fixed synaptic location, might contribute an important element to the coincidence detection machinery of the synapse.

### EXAMPLE 12

Studies of the Subcellular Localization of Calsyntenin-1 by Subcellular Fractionation and Isolation of Synaptosomes: Calsyntenin-1 is Located in the Postsynaptic Membrane, but not Anchored in the Postsynaptic Density.

To address the question whether calsyntenin-1 was firmly attached to the so-called postsynaptic density (PSD), we isolated synaptosomes by means of subcellular fractionation. For the subcellular fractionation, the protocol of Phelan and Gordon-Weeks was used (Phelan and Gordon-Weeks, 1997). Brains of 200 adult mice and 20 adult chickens, respectively, were homogenized with a Dounce homogenizer in 5 volumes of 10 mM HEPES, 0.32 M sucrose supplemented with the Mini Complete inhibitor mix (Roche). The subcellular fractionation was performed as described by Phelan and Gordon-Weeks 1997 (Isolation of synaptosomes, growth cones and their subcellular components. In: Neurochemistry—a practical approach. 2^{nd} edition. (eds. Turner A J, Bachelard H S) IRL Press, pp 1-38). For Western blot analysis with the antibodies R63 and R71, 100 μg total protein was loaded per lane. For controlling the correct fractionation, we used commercially available antibodies for the GluR1 subunit of the AMPA receptor (from Pharmingen). PSD-95 is a typical component of the postsynaptic density, whereas GluR1 is a component of the AMPA-type glutamate receptor, which has been demonstrated to exhibit a firm attachment to the PSD by a high-affinity binding site on its C-terminus (for a review: O'Brien et al., 1998). For the immunodetection of GluR1 and PSD95, 50 μg total protein were loaded per lane.

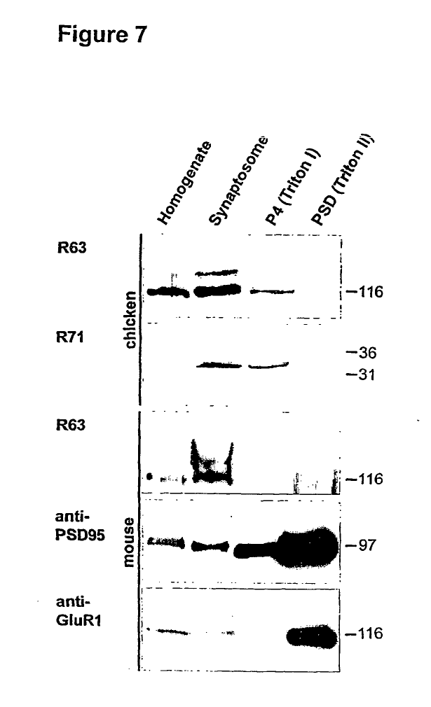

With this analysis, we found that calsyntenin-1 is a protein of th postsynaptic membrane, but does not have an intimate binding to the PSD. As demonstrated in FIG. 7, synaptosomes were enriched in full-length calsyntenin-1 and its cleavage products. Hypotonic disruption of synaptosomes and treatment with a mild detergent resulted in the solubilization of all three forms of calsyntenin-1. In contrast, typical markers of the postsynaptic density, viz. PSD-95 and GluR1 (O'Brien et al., Neuron. 1998; 21(5):1067-78), remained in the particulate fractions (P4 and PSD, according to Phelan and Gordon-Weeks 1997 (Isolation of synaptosomes, growth cones and their subcellular components. In: Neurochemistry—a practical approach. 2nd edition. (eds Turner A J, Bachelard H S) IRL Press, pp 1-38). The clearance of calsyntenin-1 from the PSD fraction indicates that its cytoplasmic segment is not firmly associated with the subsynaptic molecular scaffold that corresponds to the PSD observed in the EM and that is operationally defined as the particulate matter resulting after detergent-treatment of synaptosomes.

### EXAMPLE 13

Studies of the Localization of the Transmembrane Cleavage Product of Calsyntenin-1 by Immunoelectron Microscopy: The Transmembrane Fragment of Proteolytically Cleaved Calsyntenin-1 is Accumulated in the Spine Apparatus of Spine Synapses and the Subsynaptic Membranes of Shaft Synapses

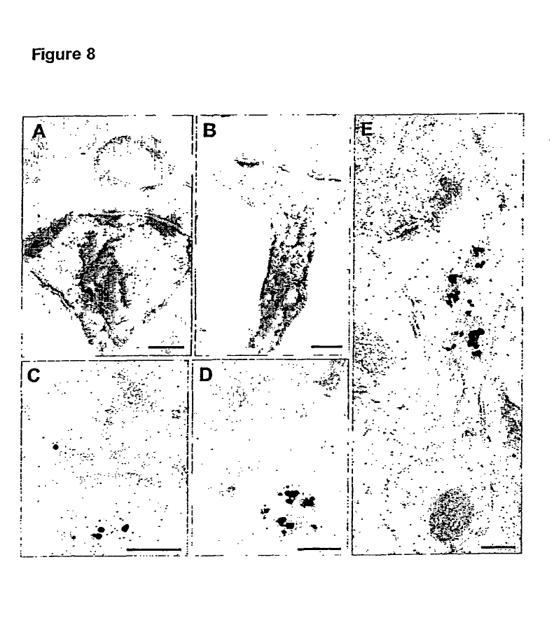

To identify the fate of the transmembrane segment of calsyntenin-1 after proteolytic cleavage, we used R71, the antibody against the membrane-proximal segment for immuno-EM with peroxidase- and gold-conjugated secondary antibodies. With peroxidase the membranes of the spine apparatus in spine synapses were labeled (FIG. 8, A and B). In some synapses, weaker immunoreactivity was also found over the postsynaptic membrane (FIG. 8A). Similarly, a strong signal was found in the lamellar membranes found beneath a fraction of the synapses in dendritic shafts and neuronal somas (not shown). With immunogold, known as less sensitive, labeling was found exclusively in association with the spine apparatus (FIG. 8, C-E) and the subsynaptic membranes of shaft synapses. Because no labeling of the subsynaptic membranous organelles was found with the N-terminal antibodies (FIG. 6), we concluded that the spine apparatus contained neither full-length calsyntenin-1 nor the N-terminal cleavage product. Therefore, the full-length as well as the 115 kD form of calsyntenin-1 found in Western blots of synaptosomes (FIG. 7) can only be derived from the postsynaptic membranes. These results indicate hat the proteolytic cleavage must occur at the cell surface, i.e. in the synaptic cleft, and that the transmembrane stump is internalized thereafter.

Proteolytic cleavage in the extracellular segment results in the release of the major extracellular portion of calsyntenin-1. This soluble fragment of calsyntenin-1 spreads in the extracellular fluids, as demonstrated by its accumulation in the cerebrospinal and the ocular vitreous fluid. The remaining transmembrane stump is internalized into the spine apparatus. Due to its membrane topology, the Ca²⁺-binding domain of the internalized transmembrane stump covers the cytoplasmic surface of the spine apparatus. Thus, internalization may translocate the calsyntenin-1-mediated Ca²⁺ buffer from the postsynaptic membrane to the surface of the spine apparatus.

Recently, the release of Ca²⁺ from intracellular stores, i.e. the ER and the spine apparatus, via activation of the IP3 receptors, was identified as an important contribution to the Ca²⁺ signal within dendritic spines (Finch and Augustine, Nature 1998; 396(6713):753-6). The IP3-mediated Ca²⁺ release is regulated by cytoplasmic Ca²⁺ in a biphasic mode (Taylor, Biochim Biophys Acta. 1998; 1436(1-2):19-33) Release is low at both low and high Ca²⁺ concentrations, but favored at intermediate concentrations of 200-300 nM. By its capacity to prolong Ca²⁺ elevations the cytoplasmic domain of calsyntenin-1 may modulate Ca²⁺ effects on IP3-mediated Ca²⁺ release.

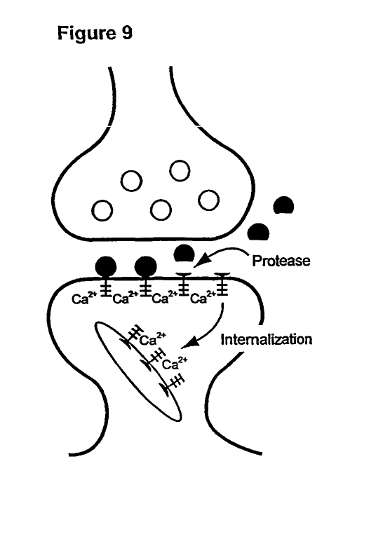

The comparison of the results of the immuno-EM analysis and subcellular fractionation indicated that the full-length form and both cleavage products of calsyntenin-1 occur in the postsynaptic membrane (for an illustration see FIG. 9). In contrast, neither full-length calsyntenin-1 nor the N-terminal cleavage product, but exclusively the transmembrane stump, was found in internal membranes. This complete segregation of the N-terminal and the C-terminal cleavage products to the interstitial fluids and to the internal membranes, respectively, can only be explained by extracellular cleavage of calsyntenin-1 by a protease located in the synaptic cleft. The selectivity of the internalization process for the transmembrane stump of calsyntenin-1 implicates a regulatory role of the proteolytic cleavage in the synaptic cleft for the translocation of the Ca²⁺-binding domain of calsyntenin-1 from the postsynaptic membrane to the surface of the spine apparatus.

### EXAMPLE 14

Determination of the Region of Calsyntenin-1 Bearing the Proteolytic Cleavage Site.

The location of the proteolytic cleavage site within the sequence of full-length calsyntenin-1 remains to be determined. Based on the location of the tryptic peptides sequenced after tryptic cleavage of the released 115 kD fragment (marked in gray in FIG. 2), the released fragment must have a length of at least 747 amino acids (as counted from the N-terminus of the mature protein). Furthermore, the cleavage occurs in the extracellular moiety, i.e. on the N-terminal side of the transmembrane segment. Thus, the cleavage site has to be located after amino acid 746 (the last amino acid of the sequenced peptide number 7) and before amino acid 834 (the first amino acid of the transmembrane segment). The segment of the extracellular moiety of calsyntenin-1 defined in this way as the peptide bearing the proteolytic cleavage site is indicated below:

Cleaved sequence of chicken calsyntenin-1 (Seq. Id. No. 29):

LIRYRNWHTVS LFDRKFKLVC SELNGRYVSN
EFKVEVNVIH TANPIEHANH IAAQPQFVHP VHHTFVDLSG
HNLANPHPFS VVPSTATV

Cleaved sequence of human calsyntenin-1 (Seq. Id. No. 30):

LLRYRNWHAR SLLDRKFKLI CSELNGRYIS
NEFKVEVNVI HTANPMEHAN HMAAQPQFVH PEHRSFVDLS
GHNLANPHPF AVVPSTATV

The first amino acid is the last amino acid of the sequenced peptide number 7 of the 115 kD fragment of calsyntenin-1 (see example 2 and FIG. 2). The last amino acid is the first amino acid of the transmembrane segment (see FIG. 2).

Neither the nature of the protease that cleaves calsyntenin-1 nor the mechanism conferring selective internalization of the transmembrane stump of calsyntenin-1 are currently not known. Several extracellular proteases have been reported to be expressed in the nervous system, including tissue plasminogen activator, thrombin, neurotrypsin, and neuropsin. For two of them, namely tPA and neuropsin, transcription has been reported to be regulated by neuronal activity (Chen et al., Neuro-chem. Int. 1995; 26(5):455-64). They are intriguing candidates for regulators of calsyntenin-1 internalization.

### EXAMPLE 15

Studies of the Subcellular Localization of Calsyntenin-1 in Rowing Neurons: Calsyntenin-1 is Found in Growth Cones of Growing Axons During Neural Development

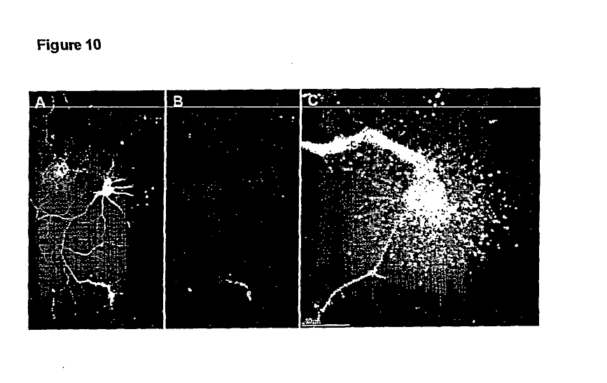

In situ hybridization indicated the expression of calsyntenin-1 mRNA in neuronal precursor cells of the germinal layers of the developing nervous system and in the early postmitotic stages of neurons in all regions of the developing nervous system. Therefore, we investigated the subcellular localization of calsyntenin-1 also in neurons during the period of cell migration and neurite growth. In this example, neurons of E18 mouse hippocampus were cultivated as described previously. When the dissociated cells from the E18 mouse hippocampus were plated at low density, their growing processes did not make contact with other cells during the first 10 days in culture. Therefore, these cultures allowed the microscopic inspection of the growth cones, the leading tips of the growing axons. As demonstrated in FIG. 10, calsyntenin-1 immunoreactivity was found on the surface of the neuronal cell soma and on both types of processes, viz. dendrites and axons. A particularly strong calsyntenin-1 immunoreactivty was found over the growth cones, as evidenced by the comparison with a double-immunofluorescence staining for calsyntenin-1 with antibody R63 (FIG. 10A) and an antibody against the axonal marker protein Tau1 (FIG. 10B). At higher magnification, calsyntenin-1 immunoreactivity in growth cones exhibited a patchy pattern, indicating that calsyntenin-1 in growth cones occurs in multiple clusters.

The results presented in this example indicate that calsyntenin-1 is abundantly expressed on the surface of growing neurons and, thus, may have a function in developmental processes, such as neuronal migration and the formation of axons and dendrites, or in nerve regenerative functions after nervous tissue injury. The particularly strong calsyntenin-1 signal found over growth cones implicates calsyntenin-1 in growth cone functions, such as axon growth and guidance.

### EXAMPLE 16

Overexpression of Calsyntenin-1 in CNS Neurons Using Transgenic Mice Technology

The overexpression of a gene in a transgenic mouse is a relatively direct way to study the function of a protein in vivo. For the first series of experiments chicken calsyntenin-1 was expressed under the control of the promoter of the Thy-1 gene. The Thy-1 gene is expressed in the nervous system relatively late (postnatal day 4-10, depending on the location). The expression of calsyntenin-1 under the control of the Thy-1 promoter (Gordon et al., 1987, Cell 50(3), 445-52) ensures that the earlier developmental stages are not affected. This point is essential. Calsyntenin-1 is expressed in some regions of the developing nervous system relatively early and, thus, it could play a role in early developmental functions, such as cell migration and axonal pathfinding. By using a late onset promoter it was intended to prevent perturbations of early stages of neurogenesis in the transgenic animals. However, depending on the aim of an investigation, other promoters may be used as well.

For the first transgenic mice, we chose to overpress the calsyntenin-1 of the chicken without the cytoplasmic segment, because of its potential of being selectively detected with species-specific monoclonal antibodies. The chicken calsyntenin-1 exhibits an amino acid sequence identity with its counterpart of the mouse of 84.7%. Thus, a highly conserved function can be assumed.

The construct of the transgene is based on an expression vector for Thy-1 in which the translated region of Thy-1 has been substituted by a Xho-I linker (Gordon et al., 1987, Cell 50(3), 445-52). The 2682 bp long DNA fragment of chicken calsyntenin-1 used for the overexpression is derived from the chicken cDNA divested with AflIII (3 bp upstream of the start ATG) and Cac8I (9 bp downstream of the TAA stop codon). This fragment is inserted into the Thy-1 expression vector at the Xho-I linker site by a blunt-end ligation and the orientation controlled with a HindIII digest. The plasmid is rescued and the fragment to be used for the injection into the pronucleus of fertilized mouse oocytes is cut out by digestion at the two flanking Pvu1 sites. The 9 kb long injection fragment is separated on a 1% agarose gel, the band purified with a QIAEXII-kit, and the DNA eluted from the QIAEX particles with injection buffer. The generation of transgenic mice was achieved by pronuclear injection following standard protocol. The litters were screened for the presence of the transgene by PCR and Southern blotting.

By this procedure, three mouse lines overexpressing the chicken calsyntenin-1 and two lines overexpressing the mouse calsyntenin-1 were raised. The expression of the transgene was verified at the mRNA level by Northern blotting and in-situ-hybridization and at the proteine level by Western blotting. A typical overexpression was in the order of 6 to 12 fold.

By the same method, transgenic animals expressing full-length calsyntenin-1, as well as other truncated forms of calsyntenin-1 or mutated forms of calsyntenin-1 (point mutations or deletion mutations) may be generated. Instead of the Thy-1 promoter, other promoters may be used, including promoters driving transgene expression in particular subpopulations of neurons, such as the promoter of the Purkinje cell-specific L7 protein or the limbic system-specific protease neuropsin. Alternatively, transgene expression may be put under the control of inducible promoters.

### EXAMPLE 17

Expression of Human Calsyntenin-1 in Eukaryotic (HEK293) Cells

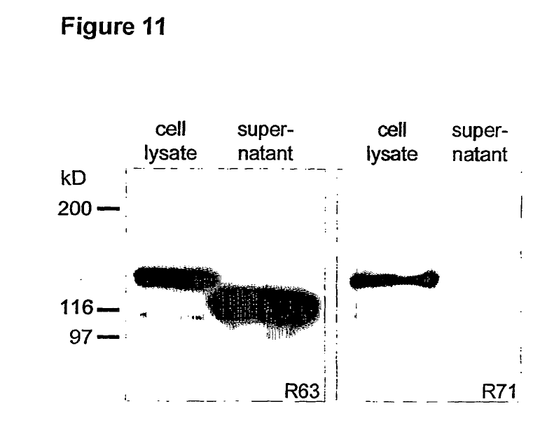

For the eukaryontic expression of human calsyntenin-1 the complete cDNA of human calsyntenin-1 was ligated into the pcDNA3.1A expression vector (Invitrogen) using the restriction sites HindIII and XbaI. The plasmid was used to transfect HEK293 cells using standard calcium phosphate transfection techniques. After 3 days the cell supernatant and cell lysate was collected, subjected to SDS-PAGE and analysed by western blotting using R63 antibody or R71 antibody. In the cell lysate of HEK293 cells the full-length human calsyntenin-1 (150 kD) was enriched whereas in the supernatant of the HEK293 cells only released human calsyntenin-1 (116 kD) was found (FIG. 11).

Expression in eucaryotic cells may, alternatively, be achieved with a variety of eucaryotic expression vectors (commercially available or self-made). A frequently used eucaryotic expression system uses vectors derived from baculovirus. For eucaryotic expression, a variety of eucaryotic cell lines may be used (such as COS cells, CHO cells, HeLa cells, H9 cells, Jurkat cells, NIH3T3 cells, C127cells, CV1 cells, or Sf cells.). For a detailed description of the use of COS cells or CHO cells, or a baculovirus-based expression system see International Application Number PCT/US96/16484 or International Publication Number WO 98/16643.

### EXAMPLE 18

Expression of Cytoplasmic Segment of Calsyntenin-1 in Eukaryotic (HEK293) Cells

For the eukaryontic expression of calsyntenin-1 a fusion protein containing the c-kappa light chain at the C-terminus of the cytoplasmic segment of chicken calsyntenin-1 was generated. As the fusion protein was expressed as a released protein in HEK293 cells the signal peptide of NgCAM was cloned at the N-terminus of chicken calsyntenin-1. The three DNA fragments of the fusion protein, signal peptide, cytoplasmic segment and c-kappa light chain were ligated in frame using restriction sites ApaL1 and HindIII. Then the cDNA of the fusion protein was ligated into pcDNA3.1 vector (Invitrogen) using restriction sites XbaI and BamHI. The plasmid was transfected into HEK293 cells using standard calcium phosphate transfection technique. After 4 days the cell supernatant was harvested and the fusion protein was purified with a 187.1 antibody coupled affinity column.

Expression in eucaryotic cells may, alternatively, be achieved with a variety of eucaryotic expression vectors (commercially available or self-made). A frequently used eucaryotic expression system uses vectors derived from baculovirus. For eucaryotic expression, a variety of eucaryotic cell lines may be used (such as COS cells, CHO cells, HeLa cells, H9 cells, Jurkat cells, NIH3T3 cells, C127 cells, CV1 cells, or Sf cells.). For a detailed description of the use of COS cells or CHO cells, or a baculovirus-based expression system see International Application Number PCT/US96/16484 or International Publication Number WO 98/16643.

### EXAMPLE 19

Cloning of the cDNA of Human Calsyntenin-2

The High Throughput Genomic Sequence database at NCBI was searched with the program tblastn using the protein sequence of the human calsyntenin-1. Twelve putative exons of a new member of the calsyntenin family were found in the sequence with the accession number AC010181, which encodes a peptide homologous to the C-terminal 713 amino acids of human calsyntenin-1.

In order to determine whether these putative exons belong to a transcribed gene, PCR primers were designed based on sequence of the second putative exon (S3eBfwd: 5′-CTCCTCTGGCATCATTGACCTC-3′) (Seq. Id. No. 31) and the last putative exon (S3rev: 5′-CATTTCTTCCTCGGCTTCTTCC-3′) (Seq. Id. No. 32). First strand cDNA was synthesized from polyA⁺ mRNA from human hippocampus (Clontech, catalog # 6578-1) with random hexamer primers using the ThermoScript RT-PCR System from GibcoBRL Life Technologies and used as template in a PCR reaction with the primers S3eBfwd and S3rev. A fragment of the expected length was obtained, subcloned into pBluescript KS+, and completely sequenced. Over large segments, the obtained sequence was identical with the predicted cDNA from the genomic sequence. However, an additional exon, that was not predicted from the genomic sequence, was found. This 1762 base pair fragment was radiolabeled and used as a probe to screen a human fetal brain cDNA library (Clontech 5′ STRETCH PLUS in λgt10, catalog # HL3003a). 10 clones were isolated, their inserts subcloned into pBluescript KS+, and completely sequenced. The clones were assembled into a contiguous cDNA. The N-terminal 116 amino acids (by homology to calsyntenin-1 ) were still missing. Therefore an EcoRI-EcoNI fragment representing the most N-terminal 400 base pairs of calsyntenin-2 was used to rescreen the same cDNA library. One clone contained 109 additional N-terminal amino acids and showed a 100% identity with a sequence on another HTGS clone, AC009671. Together with this genomic clone, we were able to assemble a full-length cDNA of the human calsyntenin-2. The N-terminal sequence was confirmed with PCR using the primers hsCst2atgfwd (5′-TGCTGCGAGGATGCTGC-3′ (Seq. Id. No. 33), containing the ATG start codon) and hCs2seq4r (5′-ATGATGCCAGAGGAGGC-3′) (Seq. Id. No. 34).

In summary, we found a single long ORF of 2865 nucleotides, encoding a protein of 955 amino acids. This cDNA was submitted to the EMBL/Genbank/DDBJ database under the name calsyntenin-2 and received the accession number AJ 278018. The protein translation of human calsyntenin-2 shows 57% identity and 67% similarity to human calsyntenin-1, and 51% identity and 59% similarity to human calsyntenin-3. Very much like calsyntenin-1, calsyntenin-2 is a type I transmembrane protein with a single transmembrane segment of 19 amino acids. The large N-terminal moiety of calsyntenin-2 is composed of 834 amino acids and located in the extracellular space. The C-terminal segment has a length of 102 amino acids and is highly enriched in acidic residues. Among the 102 residues of the cytoplasmic segment, 33 are acidic.

A high degree of sequence identity with calsyntenin-1 was also found in the region proximal to the transmembrane segment, which bears the protolytic cleavage site in calsyntenin-1. This suggests that calsyntein-2 also bears a proteolytic cleavage site in this segment.

The segment of calsyntenin-2 corresponding to the cleaved segment of calsyntenin-1 has the following amino acid sequence:

Putative cleaved sequence of human calsyntenin-2 (Seq. Id. No. 35):

HIRYRNWRPA SLEARRFRIK CSELNGRYTS NEFNLEVSIL HEDQVSDKEH VNHLIVQPPF LQSVHHPESR SSIQHSSVVP SIATV

In order to obtain information on the expression pattern of calsyntenin-2 in different human tissues, a Northern blot of poly(A)⁺ RNA from adult human tissues (Cat. Nr. 7760-1, Clontech) was hybridized with a 1762 bp cDNA fragment of human calsyntenin-2 labeled with [α-³²P] dCTP (Amersham) using the Prime-it II random primer labeling kit (Stratagene). Hybridization was performed for 2 h at 55° C. and the hybridization signals were analyzed with a PhosphorImager (Molecular Dynamics). A single species of calsyntenin-2 mRNA of approximately 5.5 kb was found. The highest expression of calsyntenin-2 mRNA was observed in brain, heart, and kidney. Low signals were detected in skeletal muscle. No transcript was found in placenta, lung and liver.

### EXAMPLE 20

Cloning of the cDNA of Human Calsyntenin-3

In a database search we found an unclassified human cDNA, KIAA0726, with a sequence identity of 53.0%, 52.3% and 54.5% with human, mouse, and chicken calsyntenin-1 , respectively (Table I). As described in the following paragraphs, we have isolated overlapping fragments matching this sequence by RT-PCR. We found a cDNA that was identical with the sequence of KIAA0726 over a large part of the ORF, but differed in the N-terminal segment. We termed this cDNA calsyntenin-3 and submitted it to the EMBL/Genbank/DDBJ database, where it was registEred with the accession number AJ277460.

The cloning of the cDNA of human calsyntenin-3 was based on a RT-PCR strategy. In a first round, we cloned the cDNA of the mouse ortholog of KIAA0726. Approximately 2×10⁶ plaques of an adult mouse BALB/c 5′ stretch plus whole brain cDNA library (ML 3000a, Clontech, Palo Alto, Calif.) were screened. Two independent clones with homology to KIAA0726 were isolated. A detailed analysis of these clones revealed a marked difference to KIAA0726 at the 5′ end of the ORF. Both clones consist of a 120 bp 5′ UTR, a translation initiation codon (ATG), and an initial part of the ORF without any homology to KIAA0726. Further downstream, however, the sequence of the clones is homologous to KIAA0726. The nucleotides adjacent to the translation codon (ATG) are in very close agreement with the consensus sequence, as determined by Kozak (Nucleic Acids Resarch 1987; 15(20): 8125-48). Due to its homology to calsyntenin-1, the novel gene of the mouse genome was termed calsyntenin-3.

Signal peptide analysis programs predicted that mouse calsyntenin-3 contains a signal peptide of 19 aa. In contrast, with the same analysis programs, no signal peptide was predicted for KIAA0726.

A screen through the human EST database revealed a human EST (expressed sequence tag; accession number: AL133677) with a nucleotide sequence identity of 79.4% with mouse calsyntenin-3. The 3′ region of EST AL133677 was identical with a seqment of KIAA0726 and exhibited a high degree of similarity with mouse calsyntenin-3. The 5′ region, however, exhibited a similarity with the 5′ end of the ORF of mouse calsyntenin-3, but was completely unrelated with any sequence of KIAA0726. The translated nucleotide sequence of EST AL133677 contains, like mouse Calsyntenin-3, a signal peptide of 19 aa length. The identity of the signal peptide of the ORF of EST AL133677 exhibited an amino acid sequence identity of 68.4% with the signal peptide of mouse calsyntenin-3. Based on these characteristics, we conlcuded that the novel sequence obtained from the products of RT-PCR and EST AL133677 is the human calsyntenin-3.

In order to obtain direct information about the 5′ region of the mRNA of human calsyntenin-3, a RT-PCR approach has been undertaken. Poly (A)⁺-selected RNA from the hippocampus of an adult human (Clontech, Palo Alto, Calif.) was chosen as a template for reverse transcription. First strand cDNA was obtained with the oligo (dT)-priming method (Thermoscript RT-PCR System of Life Technologies, Basel, Switzerland). PfuTurbo DNA Polymerase (Stratagene, La Jolla, Calif.) and the following primers were used to perform the PCR reaction:

forward: hSyn2UTRfor1 (starts at the 5′ end of EST AL133677) (Seq. Id. No. 36)

5′ CTG CAG TAG CGG GGT TG 3′

backward: RFKIA02B (ends 58 nt downstream of the TAA stop codon of KIAA0726) (Seq. Id. No. 37)

5′ TGG AGT GTC TGT TTC ACC AGG 3′

This way, the complete coding sequence plus additional 275 bp of the 5′ UTR of human calsyntenin-3 was obtained. DNA sequencing of both strands of the RT-PCR fragment confirmed a difference in the 5′ part of human calsyntenin-3 and KIAA0726. The novel cDNA of human calsyntenin-3 contains an ORF of 2868 bp that encodes a protein of 956 amino acids consisting of a signal peptide of 19 amino acids and a transmembrane domain of 23 amino acids. The N-terminal, extracellular moiety of calsyntenin-3 is composed of 845 amino acids and the C-terminal, cytoplasmic moiety has 88 amino acids. Among the 88 amino acids of the cytoplasmic segment of calsyntenin-3, 16 have acidic side chains.

A high degree of sequence identity with calsyntenin-1 was also found in the region proximal to the transmembrane segment, which bears the protolytic cleavage site in calsyntenin-1. This suggests that calsyntein-3 also bears a proteolytic cleavage site in this segment.

The segment of calsyntenin-3 corresponding to the cleaved segment of calsyntenin-1 has the following amino acid sequence:

Putative cleaved sequence of human calsyntenin-3 (Seq. Id. No. 38):

ILRQARYRLR HGAALYTRKF RLSCSEMNGR YSSNEFIVEV NVLHSMNRVA HPSHVLSSQQ FLHRGHQPPP EMAGHSLASS HRNSMIP

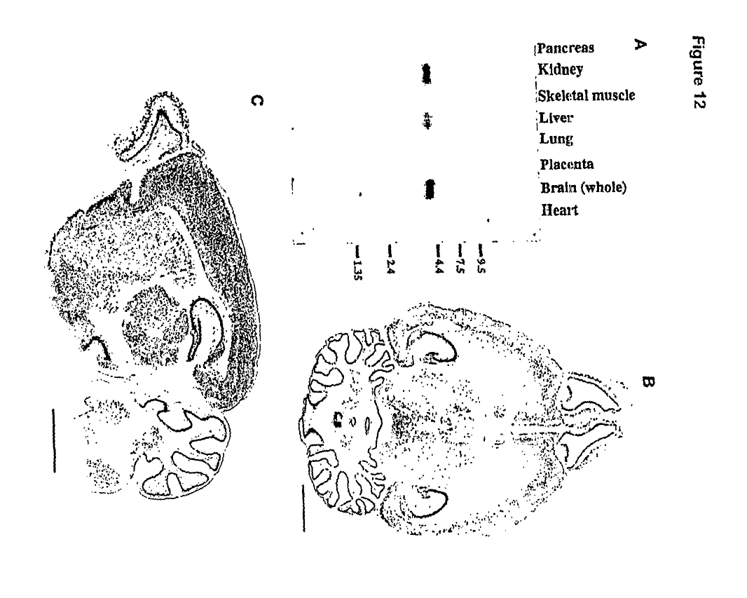

The expression pattern of calsyntenin-3 mRNA in different human tissues was determined with a commercially available human multiple-tissue Northern blot (Clontech). As a probe, a 1.15 kb cDNA fragment of human calsyntenin-3, labeled with [α-³²P] dCTP (Amersham) using the Prime-it II random primer labeling kit (Stratagene), was used. Hybridization was performed for 2 h at 42° C. and the hybridization signals were analyzed with a PhosphorImager (Molecular Dynamics). A single species of calsyntenin-3 mRNA of approximately 4 kb was revealed (FIG. 12). The highest expression of calsyntenin-3 mRNA was observed in brain. A signal of moderate intensity was found with mRNA from kidney. Low level signals were detected in pancreas, liver, heart, placenta, skeletal muscle, and lung.

The cellular resolution of the expression of calsyntenin-3 was determined by in situ hybridization, performed as described previously (Schaeren-Wiemers and Gerfin-Moser, Histochemistry. 1993; 100(6):431-4). On cryosections from an adult mouse brain, calsyntenin-3 mRNA was abundant in all areas of the gray matter. Inspection at higher magnification indicated a neuronal expression pattern in all areas of the CNS and the PNS. However, not all neurons expressed calsyntenin-3 mRNA and considerable differences in the expression levels were found. A very prominent example of the cell-type specific expression of calsyntenin-3 is found in the cerebellum. As shown in FIG. 12, cerebellular Purkinje cells exhibit a very strong in situ hybridization signal for calsyntenin-3, whereas all other cells of the cerebellum do not express detectable levels of calsyntenin-3 mRNA.

### EXAMPLE 21

Binding of the Cytosolic Segment of Calsyntenin-1 and the Arp2/3 Complex.

While studying the scientific literature dealing with interactions between cell surface proteins and the cellular cytoskeleton, we found that the cytoplasmic part of all calsyntenin family proteins (i.e. calsyntenin-1, calsyntenin-2, and calsyntenin-3) contains at least one intriguing conserved amino acid sequence motif. This motiv consists in an acidic amino acid sequence containing a conserved tryptophan that exhibits a high degree of similarity with acidic amino acid motifs containing a conserved tryptophan in the Arp2/3 binding domain of most, if not all, of the currently known activators of the Arp2/3 complex. The acidic amino acid sequence containing a conserved tryptophan is found twice in the cytoplasmic segment of calsyntenin-1, once with the amino acid sequence ..MDWDDS.. and once with ..LEWDDS.. (amino acid sequence given in single letter code). The cytoplasmic sequence of calsyntenin-2 contains one ..MDWDDS.. and one ..LEWDDS.., and the cytoplasmic segment of calsyntenin-3 contains a single motif. of this kind, namely ..LFWDDS... The Arp2/3 complex plays a central role in the regulation of the actin-based cellular motility, by regulating actin filament growth and branching (for reviews see: Borisy and Svitkina, Curr. Opin. Cell Biol. 12: 104-112, 2000; Pantaloni et al., Science 292: 1502-1506; Higgs and Pollard, Annu. Rev. Biochem., 70: 649-676, 2001; and references therein). Arp2/3 activators containing a similar acidic motif with a conserved tryptophan include human WASP (Abbreviation for: Wiscott Aldrich Syndrome Protein), the related human N-WASP, the human Scar/WAVE1 proteins, and cortactin, exhibiting the sequences ..DDEWDD, ..DDEWED and ..EVDWLE, and ..ADDWET.., respectively (for WASP, N-WASP, and Scar/WAVE1 see Higgs and Pollard, Annu. Rev. Biochem. 70: 649-676, 2001; for cortactin see Uruno et al., Nature Cell Biol. 3: 259-266, 2001). The importance of the conserved tryptophan and the adjacent acidic amino acids for Arp2/3 binding and the Arp2/3 function in actin poymerization has been demonstrated by site-directed mutagenesis of cortactin (Uruno et al., Nature Cell Biol. 3: 259-266, 2001). Site directed mutagenis of both the tryptophan and the two amino acid residues preceding the tryptophan in the sequence ..ADDWET.. resulted in the loss of Arp2/3 binding and Arp2/3-mediated actin polymerization. All these Arp2/3 activator proteins are resident in the cytoplasm and have been reported to link intracellular signals generated by the transmembrane signaling of receptors for extracellular regulators, such as growth factor, cytokines, etc., into activation of the Arp2/3 complex. A crucial intermediate step in the signaling cascade from activated transmembrane receptors to the activation of the Arp2/3 activators has been attributed to the small GTP-binding proteins of the Rho family (for a review: Takai et al., Physiol. Rev. 81:153-207, 2001). Activated Arp2/3 complex in turn initiates the generation of new actin filaments and the branching of pre-existing actin filaments (for reviews see: Borisy and Svitkina, Curr. Opin. Cell Biol. 12: 104-112, 2000; Pantaloni et al., Science 292: 1502-1506; Higgs and Pollard, Annu. Rev. Biochem., 70: 649-676, 2001; and references therein). As a result of the enhanced cytoskeletal dynamics, the cells generate and/or retract plasma membrane protrusions, such as filopodia and lamellipodia (Borisy and Svitkina, Curr. Opin. Cell Biol. 12: 104-112, 2000). In the growing tip of the axons growing out of neurons, termed growth cones, the enhanced activity so generated translates into an enhanced exploratory activity and enhanced axon growth and pathfinding activity (Hu and Reichardt, Neuron 22, 419-422, 1999; Suter and Forscher, Curr. Opin. Neurobiol. 8: 106-116, 1998; Dickson, Curr. Opin. Neurobiol. 11: 103-110, 2001). The enhanced dynamics of actin filaments in the dendritic spines of neurons of the central nervous system results in an enhanced motility, which in turn may regulate the morphological shape and the electrical properties of the spine. As a consequence, the postsynaptic response to presynaptic signals may be altered (Segal et al., Trends Neurosci. 23: 53-57, 2000; Halpain, Trends Neurosci. 23: 141-146, 2000; Matus, Science 290: 754-758, 2000; Scott and Luo, Nature Neurosci. 4: 359-365, 2001). In non-neuronal cells, the enhanced dynamics of actin filaments induced via Arp2/3 activation results in an increase in cell motility, accompanied by a boost in the formation of membrane protrusions, such as lamellipodia, and enhanced migratory activity (Holt and Koffer, Trends Cell Biol. 11: 38-47, 2001; Mullins, Curr. Opin. Cell Biol. 12: 91-96, 2000; Prokopenko et al., J. Cell Biol. 148: 843-848, 2000). A dysregulated signalling from the cell surface to the cytoskeleton, resulting in altered cell motility, enhanced formation of lamellipodia, and enhanced locomotion, when found in tumour cells, strengthens the capacity of the tumor cells for invasive growth and metastasis (Radisky et al., Seminars Cancer Biol. 11:87-95, 2001; Kassis et al., Seminars Cancer Biol. 11:105-119, 2001; Condeelis et al., Seminars Cancer Biol. 11:119-128, 2001; Price and Collard, Seminars Cancer Biol. 11:167-173, 2001).

The acidic sequence containing a tryptophan residue was also found to be crucial for the induction and the branching of actin filaments generated Listeria monocytogenes (Higgs and Pollard, Annu. Rev. Biochem., 70: 649-676, 2001; Cameron et al., Curr. Biol. 11: 130-135, 2001). After invading the cytosol of the host cell, these bacteria use the cellular actin machinery for their own locomotion. The bacterial surface protein ActA initiates the formation of actin filaments on the surface of Listeria monocytogenes. Very much like the cells own Arp2/3 activators, ActA of Listeria monocytogenes contains a tryptophan flanked by acidic residue. It has the amino acid sequence ..DEWEE.. (for a review: Higgs and Pollard, Annu. Rev. Biochem. 70: 649-676, 2001).

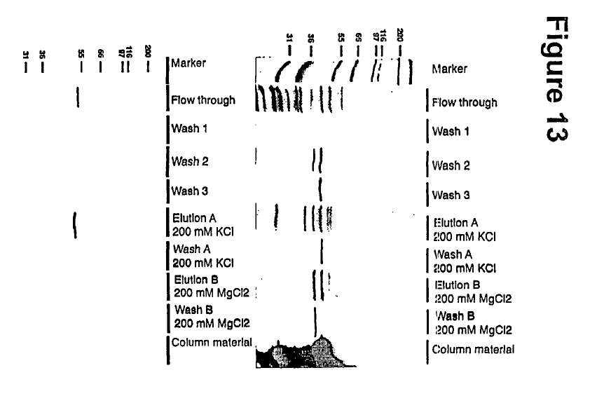

Based on the high degree of similarity with the Arp2/3-binding and Arp2/3-activating motif of the currently published Arp2/3 activators, we speculated that the proteins of the calsyntenin family may regulate the dynamics of the actin cytoskeleton via binding to and regulating of the function of the Arp2/3 complex. To test this hypothesis, we generated a fusion protein composed of glutathion-S-transferase (GST) and the cytoplasmic segment of calsyntenin-1 (GST-Cst_{C}). The COOH-terminal domain (Cst_{C}) of human calsyntenin-1 was expressed and purified as a GST fusion protein in E. coli. The region encoding the cytoplasmic segment of human calsyntenin-1 (Cst_{C}: residues 881-981) was amplified by PCR from human brain cDNA using oligonucleotides hsCst1-881f (5′-CGGGATCCCGCATCCGGGCCGCACAT-3′) (Seq. Id. No. 39) and hsCst1-981r (5′-GGGAATTCCTCAGTAGCTGAGGGTGGAG-3′) (Seq. Id. No. 40) as forward and reverse primer, respectively. The Cst_{C} PCR fragment was cloned into the BamH1/EcoR1 sites of the pGEX6P-1 plasmid. The resulting pGEX-GST-Cst_{C} plasmid was transfected into the E. coli strain BL21 and the expression of GST-Cst_{C} protein was induced according to standard procedures. GST-Cst_{C} fusion protein was purified according to standard procedures using Glutathione-Sepharose (Amersham Pharmacia Biotech) and kept at 4° C. until use. To generate an affinity column, 3.5 mg of GST-Cst_{C} were bound to 0.75 ml Glutathion-Sepharose by batch incubation. Thereafter, the GST-Cst_{C} conjugated Glutathion-Sepharose was packed into a column and equilibrated with Buffer B (see below). Bovine brain extract was prepared according to the procedure described previously (Uruno et al., Nature Cell Biol. 3:259-266, 2001). Briefly, 100 g of frozen bovine brain were minced with a Waring blender in 100 ml of buffer Q (20 mM Tris, 100 mM NaCl, 5 mM MgC12, 5 mM EGTA and 1 mM dithiothreitol (DTT), pH 8.0), supplemented with 50 μg/ml phenylmethylsulphonyl fluoride, 5 μg/ml leupeptin and 1 μg/ml aprotinin. The minced tissue was further homogenized using a Dounce homogenizer and was clarified by centrifugation at 10,000 g for 60 min at 4° C. The supernatant was subjected to chromatography in a 100-ml Q Sepharose Fast-flow column equilibrated with buffer Q. The flow-through was collected, supplemented with 0.1 mM ATP, and loaded on a GST-Cst_{C} Glutathione-Sepharose column equilibrated with buffer B (50 mM Tris, 25 mM KCl, 1 mM MgCl2, 0.5 mM EDTA, 1 mM DTT, 0.1 mM ATP, pH 7.5). After washing with buffer B, elution was initiated with 0.2 M KCl in buffer B, followed by a second elution with buffer B containing 0.2 M MgCl2. The eluted protein was analyzed by SDS-PAGE followed by Western blotting using a commercially available antibody against the Arp3 subunit of the Arp2/3 complex (Santa Cruz). As demonstrated in FIG. 13, the Arp2/3 complex, contained in the complex mixture of proteins extracted from bovine brain, bound to the immobilized GST-Cst_{C} fusion protein and was released only when elution conditions were applied (Elution A in FIG. 13). Nonbinding of Arp2/3 was observed when bovine brain extract was passed over a column containing only the GST part. This indicates that the observed binding of Arp2/3 to the GST-Cst_{C} fusion protein is mediated by the cytoplasmic segment of calsyntenin-1. Based on the presence of highly similar segments containing a conserved tryptophan flanked by acidic amino acids in the cytoplasmic parts of calsyntenin-2 and calsyntenin-3 all members of the calsyntenin family bind to and, thus, regulate Arp2/3 activity.

To demonstrate direct binding between the cytoplasmic segment of calsyntenin-1 and the Arp2/3 complex, Arp2/3 complex was purified according to a published protocol (Egile et al., J. Cell Biol. 146:1319-1332, 1999). To prepare an affinity ligand for Asp2/3 complex, the COOH-terminal domain (VCA) of human N-WASP was expressed as a GST fusion protein in E. coli and purified using a Glutathion-Sepharose column. The region encoding the VCA segment of human N-WASP (residues 392-505) was amplified by PCR from human brain cDNA using oligonucleotides phNW392 (5′-ccggaattcCCTTCTGATGGGGACCATCAG-3′) (Seq. Id. No. 41) and phNW505 (5′-ccgctcgagTCAGTCTTCCCACTCAT CATC-3′) (Seq. Id. No. 42) as forward and reverse primer, respectively, as described previously (Egile et al., J. Cell Biol. 146:1319-1332, 1999). The PCR fragment encoding N-WASP VCA was cloned into the XhoI site of the pGEX6P-1 plasmid, to generate the pGEX-VCA plasmid. GST-VCA protein was expressed in the E. coli strain BL21 according to standard induction and purification procedures. GST-VCA fusion protein was purified on a Glutathion-Sepharose column and eluted following the protocol recommended by the supplier (Amersham Pharmacia Biotech). To generate an affinity column, purified GST-VCA was bound to Glutathion-Sepharose beads by batch incubation. GST-VCA glutathione Sepharose beads were stored at 4° C. until use.

The GST-VCA Glutathion-Sepharose was used as the affinity matrix to purify the Arp2/3 complex, as described previously (Uruno et al., Nature Cell Biol. 3:259-266, 2001). Briefly, 100 g of frozen bovine brain were minced with a Waring blender in 100 ml buffer Q (20 mM Tris, 100 mM NaCl, 5 mM MgCl2, 5 mM EGTA, 1 mM dithiothreitol, pH 8.0) supplemented with 50 μg/ml phenylmethylsulphonyl fluoride, 5 μg/ml leupeptin and 1 μg/ml aprotinin. The minced tissue was further homogenized using a Dounce homogenizer and was clarified by centrifugation at 10,000 g for 60 min at 4° C. The supernatant was subjected to chromatography in a 100-ml Q Sepharose Fast-flow column equilibrated with buffer Q. The flow-through containing the Arp2/3 complex was collected, supplemented with 0.1 mM ATP and fractionated on a GST-VCA glutathione-sepharose column equilibrated with buffer B (50 mM Tris, 25 mM KCl, 1 mM MgCl2, 0.5 mM EDTA, 1 mM DTT, 0.1 mM ATP, pH 7.5). After washing with 0.2 KCl in buffer B, the Arp2/3 complex was eluted with buffer B containing 0.2 M MgCl2. The protein was then dialysed against buffer B and concentrated with a Centriprep 10 cartridge. The concentrated Arp2/3 complex was stored in buffer B containing 30% glycerol at −80° C. Protein concentration was determined by the BCA method (Pierce protein assay), using BSA as a standard.

To investigate whether the cytoplasmic segment of calsyntenin-1 has the capacity to bind the Arp2/3 complex, the GST-Cst_{C} fusion protein was bound to Glutathion-Sepharose beads by batch incubation. For a control, the GST-VCA fusion protein, which is an established ligand of the Arp2/3 complex (Uruno et al., Nature Cell Biol. 3:259-266, 2001) and GST alone were bound to Glutathion-Sepharose. GST-Cst_{C}, GST-VCA, or GST (5 μg), immobilized on Glutation-Sepharose beads, were mixed with 10 pmol of purified Arp2/3 complex in buffer A (100 82 l of 50 mM Tris, 1% Triton-X-100), and incubated for 2 h at 4° C. on a rotating wheel. The beads were rinsed three times with buffer A and then boiled in two times SDS sample buffer. The resulting sample buffer was loaded on an SDS-PAGE gel. The electrophoretically separated proteins were electrotransferred onto nitrocellulose using standard protocols. The Arp2/3 complex was visualized with a polyclonal anti-Arp3 antibody (purchased from Santa Cruz) according to standard immunoblotting procedures. We found unequivocal evidence for direct binding of Arp2/3 to GST-Cst_{C} and GST-VCA, but not GST alone. These results demonstrate a direct binding interaction between the cytoplasmic segment of calsyntenin-1 and the Arp2/3 complex.

In summary, calsyntenin-1 containing the cytoplasmic sequences ..MDWDDS.. and ..LEWDDS.. is capable of binding the Arp2/3 complex. This indicates that calsyntenin-1 uses the same binding site to interact with the Arp2/3 complex at as the currently known regulators of Arp2/3 activity, including human WASP, human N-WASP, the human Scar/WAVE1 proteins, cortactin, and the ActA protein of Listeria monocytogenes, in which the binding site comprising the conserved tryptophan includes the sequences ..DEWDD, ..DEWED, ..VDWLE, ..ADDWET.., and ..DEWEE, respectively (for an overview see: Higgs and Pollard, Annu. Rev. Biochem. 70: 649-676, 2001 and references therein). Thus, calsyntenin-1, by means of its cytoplasmic part competes with established regulators of Arp2/3 activity and, in doing so, takes part in the regulation of Arp2/3 activity. Calsyntenin-1 is the first regulator of the Arp2/3 complex that is a transmembrane protein. In contrast, the currently known Arp2/3 regulators, including WASP, N-WASP, the proteins of the Scar/WAVE1 family, and cortactin are cytoplasmic proteins and depend on other intracellular mediators of extracellular signals. Calsyntenin-1 may transduce extracellular signals received by ist extracellular part directly into an activity-regulating signals to the Arp2/3 complex. The presence of highly similar conserved acidic segments containing a conserved tryptophan in the cytoplasmic parts of calsyntenin-2 and calsyntenin-3 indicates that all members of the calsyntenin family members bind to and, thus, regulate Arp2/3 activity.

While there are shown and described presently preferred embodiments of the invention, it is to be distinctly understood that the invention is not limited thereto but may be otherwise variously embodied and practiced within the scope of the following claims.

1. An isolated polypeptide for the use as a pharmaceutical comprising an amino acid sequence at least 50% identical to a sequence selected from the group consisting of:
a) a full length amino acid sequence selected from the group consisting of Seq. Id. No. 2, Seq. Id. No. 4 and Seq. Id. No. 6,
b) a polypeptide comprising at least one, preferably two, most preferably three of amino acid sequences selected from the group consisting of: sequence of residues from about 46 to about 165, sequence of residues 166 to about 257, sequence of residues from about 774 to about 861 and sequence of residues from about 881 to about 981 of Seq. Id. No. 2,
c) a polypeptide comprising at least one, preferably two, most preferably three of amino acid sequences selected from the group consisting of: sequence of residues from about 66 to about 158, sequence of residues from about 182 to about 259, sequence of residues from about 751 to about 834 and sequence of residues from about 854 to about 955 of Seq. Id. No. 4,
d) a polypeptide comprising at least one, preferably two, most preferably three of amino acid sequences selected from the group consisting of: sequence of residues from about 51 to about 142, sequence of residues from about 167 to about 244, sequence of residues from about 759 to about 845 and sequence of residues from about 869 to about 956 of Seq Id. No. 6
e) a polypeptide comprising at least one, preferably two, most preferably three of amino acid sequences selected from the group consisting of: sequence of residues from about 881 to about 981 of Seq. Id. No. 2, sequence of residues from about 854 to about 955 of Seq. Id. No. 4 and sequence of residues from about 869 to about 956 of Seq Id. No. 6,
and having calcium binding activity and/or the capacity to bind to the Arp2/3 complex.

2. The polypeptide of claim 1 wherein the polypeptide sequence is at least 60% identical and more preferably more than 65% identical to an amino acid sequence selected from the group consisting of:
a) a full length amino acid sequence selected from the group consisting of Seq. Id. No. 2, Seq. Id. No. 4 and Seq. Id. No. 6
b) a polypeptide comprising at least one, preferably two, most preferably three of amino acid sequences selected from the group consisting of: sequence of residues from about 46 to about 165, sequence of residues 166 to about 257, sequence of residues from about 774 to about 861 and sequence of residues from about 881 to about 981 of Seq. Id. No. 2,
c) a polypeptide comprising at least one, preferably two, most preferably three of amino acid sequences selected from the group consisting of: sequence of residues from about 66 to about 158, sequence of residues from about 182 to about 259, sequence of residues from about 751 to about 834 and sequence of residues from about 854 to about 955 of Seq. Id. No. 4,
d) a polypeptide comprising at least one, preferably two, most preferably three of amino acid sequences selected from the group consisting of: sequence of residues from about 51 to about 142, sequence of residues from about 167 to about 244, sequence of residues from about 759 to about 845 and sequence of residues from about 869 to about 956 of Seq Id. No. 6,
e) a polypeptide comprising at least one, preferably two, most preferably three of amino acid sequences selected from the group consisting of: sequence of residues from about 881 to about 981 of Seq. Id. No. 2, sequence of residues from about 854 to about 955 of Seq. Id. No. 4 and sequence of residues from about 869 to about 956 of Seq Id. No. 6,
and having calcium binding activity and/or the capacity to bind to the Arp2/3 complex.

3. An isolated polypeptide for the use as a pharmaceutical comprising an amino acid sequence selected from
sequences comprising a stretch of at least 100 amino acids with a minimal identity percentage of 50%, preferably 55% and more preferably 60% to a amino acid sequence selected from the group consisting of Seq. Id. No. 2, Seq. Id. No. 4 and Seq. Id. No. 6, said sequences having calcium binding activity and/or the capacity to bind to the Arp2/3 complex.

4. The polypeptide according to anyone of the preceding claims which
is a transmembrane protein and which
is expressed predominatly in cells of the nervous system.

5. The polypeptide according to claim 4 which is expressed in neurons.

6. The polypeptide according to claim 5 which is localised to the postsynaptic membrane of synapses.

7. The polypeptide according to claim 6 which is localized in a membrane of a spine apparatus of spine synapses and in a membrane of subsynaptic endoplasmatic reticulum of shaft synapses.

8. The polypeptide according to anyone of claims 4 to 7 which is expressed in tumor cells.

9. The polypeptide according to anyone of the preceding claims which has its major calcium-binding domain in the cytoplasmic compartment.

10. The polypeptide according to anyone of the preceding claims which has at least one binding site for the Arp2/3 complex.

11. An isolated nucleotide sequence encoding a polypeptide according to anyone of the preceding claims for the use as pharmaceutical.

12. An isolated nucleotide sequence encoding a polypeptide as defined in anyone of the preceding claims which has, due to at least one point mutation, insertion or deletion, lost its function.

13. The nucleotide sequence according to claim 12 for the use as a diagnostic tool.

14. A pharmaceutical composition comprising a polypeptide according to anyone of claims 1 to 10.

15. Pharmaceutical composition comprising a polypeptide as defined in anyone of claims 1 to 10 and/or a nucleotide sequence according to claim 11.

16. Use of a polypeptide or a partial sequence thereof as defined in anyone of the preceding claims for the use as a tool for the development of a pharmaceutical.

17. Use of a protein or a DNA sequence as defined in anyone of the preceding claims for the screening and for the preparation of a medicament for the treatment of disorders, in particular disorders of the nervous system, more particular of the central nervous system, most preferably the brain.

18. Use according to claim 17, characterized in that said disorders, in particular of the nervous system, more particular of the brain, are disorders due to lack of cleavage or miscleavage or excessive cleavage of a protein as defined in one of claims 1-11 induced by at least one protease.

19. Use according to claim 18, characterized in that the protease is tissue-type plasminogen activator, abbreviated as tPA, urokinase-type plasminogen activator, abbreviated as uPA, or plasmin or neurotrypsin, or thrombin, or neuropsin.

20. Use according to anyone of claims 17 to 19, characterized in that said disorders, in particular of the nervous system, are due to perturbed processing of intracellular calcium signals.

21. Use according to claim anyone of claims 17 to 20, characterized in that said disorders, in particular of the nervous system, are due to perturbed processing of extracellular signals that regulate the cellular motility processes by means of regulating the activity of the Arp2/3 complex.

22. Use according to one of claims 17 to 21, characterized in that the medicament is a medicament for the minimization of the tissue destruction during and/or

23. Use according to anyone of claims 17 to 22, characterized in that the medicament prevents the cell death of cells of the nervous system.

24. Use of a DNA sequence or a protein as defined in anyone of claims 1-10 for the preparation of a medicament for the treatment of tumors, including prevention or reduction of the growth, the expansion, the infiltration and the metastasis of primary and metastatic tumors, in particular brain tumors or tumors of the retina.

25. Use according to claim 24, characterized in that said tumors involve in their growth, expansion, infiltration, metastasis and promotion of blood vessels or neoangiogenesis an enhanced activity of the Arp2/3 complex.

26. Use according to claim 25, characterized in that said enhanced activity of the Arp2/3 complex is mediated by an abnormal or excessive or reduced regulatory function of one of the sequences as defined in anyone of claima 1-10.

27. Use according to anyone of claims 24 to 26, characterized in hat said tumors involve in their growth, expansion, infiltration, metastasis and promotion of blood vessels or neoangiogenesis at least one protease functionally connected with a polypeptide as defined in anyone of the claims 1-10.

28. Use according to claim 27, characterized in that the protease is a member of one of the following protease families:
Serine Protease family such as tissue-type plasminogen activator (tPA), urokinase-type plasminogen activator (uPA), plasmin, thrombin, neurotrypsin, neuropsin, elastases, cathepsin G,
Matrix Metalloproteinases family such as collagenases, gelatinases, stromelysins, matrylisins,
Cystein Proteases family such as cathepsin B and cathepsin D.

29. A method for the production of polypeptides as defined in anyone of claims 1 to 10 or such polypeptide expressing cells, characterized in that suitable host cells are transfected with a DNA sequence as defined in claim 12 in a vector ensuring the expression of said DNA sequence in said host cell, and in that said transfected cells are cultured under suitable conditions allowing said expression.

30. A synthetic or chemical method for the production of polypeptides, peptides or nucleic acid sequences representing at least part of the sequences defined in claims 1 to 13 and having the ability to mimic or to block, respectively, the biological activity of calsyntenin, in particular the calcium binding activity.

31. Use of the DNA sequences and/or the polypeptides as defined in anyone of claims 1 to 13 as tools in the screening of pharmaceutical drugs.

32. Use of a sequence as defined in claim 11 as a means to produce antigens or as antigen for the production of antibodies.

33. Transgenic non human animal, characterized in that it comprises an exogenous DNA sequence as defined in claim 12 in an environment allowing protein expression.

34. Use of a DNA sequence as defined in claim 12 or 13 or fragments thereof for the preparation of a diagnostic preparation for the diagnosis of disorders due to defects in the genomic sequence comprising a DNA sequence according to claim 11.

35. A vector or artificial chromosome comprising a DNA sequence as defined in claim 12 for the use in gene therapeutical applications in humans and in animals.

36. An isolated polypeptide comprising an amino acid sequence which is at least 60% identical to the amino acid sequence of Seq.Id. No. 4.

37. The polypeptide of claim 36 wherein the amino acid sequence is identical to Seq. Id. No. 4.

38. An isolated polypeptide comprising an amino acid sequence which is at least 98.5% identical to the amino acid sequence of Seq. Id. No. 6.

39 The polypeptide of claim 37 which is identical to Seq. Id. No. 6.

40. A nucleotide sequence encoding a polypeptide according to anyone of claims 36 to 39.

41. A protease which cleaves a polypeptide as defined in anyone of claims 1 to 10 in its extracellular part.

42. A cell extract comprising a protease which cleaves a polypeptide as defined in anyone of claims 1 to 10.
